SEQUENCE LISTING (38 sequences)

SEQ ID NO: 1 - protein, 7 aa, Homo sapiens
  MISC_FEATURE (4)..(4): Where Xaa may be C or T
  MISC_FEATURE (5)..(5): Where Xaa may be V, I, or P
GGYXXSK

SEQ ID NO: 2 - protein, 7 aa, Homo sapiens
GGYCVSK

SEQ ID NO: 3 - protein, 7 aa, Homo sapiens
GGYCISK

SEQ ID NO: 4 - protein, 7 aa, Homo sapiens
GGYTPSK

SEQ ID NO: 5 - DNA, 42 bp, Homo sapiens
gggaagagttgctttctttgtatatggagtggaagccttttc

SEQ ID NO: 6 - DNA, 42 bp, Homo sapiens
gaaaaggcttccactccatatacaaagaaagcaactcttccc

SEQ ID NO: 7 - DNA, 33 bp, Homo sapiens
ggcttctgtgtctccaggtatggagtggaagcc

SEQ ID NO: 8 - DNA, 33 bp, Homo sapiens
ggcttccactccatacctggagacacagaagcc

SEQ ID NO: 9 - DNA, 34 bp, Homo sapiens
gggtgtcactttttggttatggcgtggaagcctt

SEQ ID NO: 10 - DNA, 34 bp, Homo sapiens
aaggcttccacgccataaccaaaaagtgacaccc

SEQ ID NO: 11 - DNA, 31 bp, Homo sapiens
gcttctgcatctccaggtatggcgtggaagc

SEQ ID NO: 12 - DNA, 31 bp, Homo sapiens
gcttccacgccatacctggagatgcagaagc

SEQ ID NO: 13 - DNA, 36 bp, Homo sapiens
cgccttgcaatcgttggatatgcagtggaaggtttc

SEQ ID NO: 14 - DNA, 36 bp, Homo sapiens
gaaaccttccactgcatatccaacgattgcaaggcg

SEQ ID NO: 15 - DNA, 34 bp, Homo sapiens; identical to SEQ ID NO: 10

SEQ ID NO: 16 - DNA, 35 bp, Homo sapiens
aaccttccactgcatatctggatggagtaaagccc

SEQ ID NO: 17 - DNA, 30 bp, Homo sapiens
ctggcagccaatggttttggcctggaggcc

SEQ ID NO: 18 - DNA, 30 bp, Homo sapiens
ggcctccaggccaaaaccattggctgccag

SEQ ID NO: 19 - DNA, 31 bp, Homo sapiens
gcttctgtgtctccagatttggcctggaggc

SEQ ID NO: 20 - DNA, 31 bp, Homo sapiens
gcctccaggccaaatctggagacacagaagc

SEQ ID NO: 21 - DNA, 24 bp, Homo sapiens
agcatgcttcctttggtgaggaga

SEQ ID NO: 22 - DNA, 24 bp, Homo sapiens
ttcccgttctgaagtagccaggtt

SEQ ID NO: 23 - DNA, 24 bp, Homo sapiens
tgtggtcaacgtctccagtgtcat

SEQ ID NO: 24 - DNA, 24 bp, Homo sapiens
agagaaggcttccacgccatactt

SEQ ID NO: 25 - DNA, 22 bp, Homo sapiens
gtcaagaaagctcaagggagag

SEQ ID NO: 26 - DNA, 23 bp, Homo sapiens
ccactgcatatttggatggagta

SEQ ID NO: 27 - DNA, 19 bp, Homo sapiens
ggcgggatgtagctcattt

SEQ ID NO: 28 - DNA, 20 bp, Homo sapiens
ttctccagactctccaggtt

SEQ ID NO: 29 - DNA, 23 bp, Homo sapiens
ttctgtacctgtaacggctattt

SEQ ID NO: 30 - DNA, 21 bp, Homo sapiens
gggatctgttacccagtcatc

SEQ ID NO: 31 - DNA, 20 bp, Homo sapiens
ccttctgcactggaaatgga

SEQ ID NO: 32 - DNA, 21 bp, Homo sapiens
cacccaagctaaaccgtatgt

SEQ ID NO: 33 - DNA, 23 bp, Homo sapiens
ggtcatctgcccatcagtataag

SEQ ID NO: 34 - DNA, 21 bp, Homo sapiens
ccaaatgggatcctgtggtta

SEQ ID NO: 35 - DNA, 21 bp, Homo sapiens
cggctaatgggtggaatctaa

SEQ ID NO: 36 - DNA, 21 bp, Homo sapiens
ggttacaccaaagggctagaa

SEQ ID NO: 37 - DNA, 23 bp, Homo sapiens
gacttgtagagagacagggtaga

SEQ ID NO: 38 - DNA, 20 bp, Homo sapiens
taggagggctggcaacttag

(19) United States
(12) Patent Application Publication
(10) Pub. No.: US 2020/0179408 A1
(43) Pub. Date: Jun. 11, 2020

(54) INHIBITION OF CATALYTIC SITE COMMON TO MULTIPLE 3 ALPHA-OXIDOREDUCTASES FOR TREATMENT OF PROSTATE CANCER

(71) Applicant: Health Research, Inc., Buffalo, NY (US)

(72) Inventors: James L. MOHLER, Buffalo, NY (US); Michael FIANDALO, Buffalo, NY (US); David WATT, Lexington, KY (US); Elizabeth WILSON, Chapel Hill, NC (US)

(21) Appl. No.: 16/088,224

(22) PCT Filed: Mar. 27, 2017
(86) PCT No.: PCT/US2017/024322

§ 371 Date: Sep. 25, 2018

Related U.S. Application Data
(60) Provisional application No. 62/313,261, filed on Mar. 25, 2016.

(51) Int. Cl. A61K 31/58

(52) U.S. Cl. CPC A61K 31/58

(57) ABSTRACT

Methods and compositions which are directed against catalytic site common to multiple 3 alpha-oxidoreductases. The compositions can be used for the treatment of prostate cancer.

### CROSS-REFERENCE TO RELATED APPLICATIONS

This application claims priority to U.S. Provisional Patent application No. 62/313,261, filed on Mar. 25, 2016, the disclosure of which is incorporated herein by reference.

### STATEMENT REGARDING FEDERALLY-SPONSORED RESEARCH

This invention was made with government support under grant numbers CA016056 and CA77739 awarded by the National Cancer Institute. The government has certain rights in the invention.

### BACKGROUND OF THE DISCLOSURE

Prostate cancer (CaP) growth and progression rely on the interaction between androgen receptor (AR) and the testicular androgens testosterone (T) and dihydrotestosterone (DHT). Almost all men who present with CaP and some men who fail potentially curative therapy are treated with androgen deprivation therapy (ADT). ADT lowers circulating T levels, deprives AR of ligand and induces CaP regression. However, ADT is not curative, intratumoral androgen levels are sufficient to activate AR and CaP recurs as lethal castration-recurrent/resistant CaP (CRPC). One mechanism that may contribute to CaP resistance to ADT is intratumoral intracrine androgen metabolism, which is defined as the conversion of weak adrenal androgens to T or DHT. There are 3 androgen metabolism pathways to DHT. The frontdoor pathway uses adrenal androgens dehydroepiandrosterone (DHEA) or androstenedione (ASD), to generate T, which is 5α-reduced to DHT by 5α-reductases (SRD5A). The backdoor pathways are defined by DHT generation without using T as substrate. Previous work from our laboratory and others has demonstrated that CaP cells use the primary or secondary backdoor androgen metabolism pathways to produce DHT. The terminal step in the primary backdoor pathway involves conversion of 5α-androstane-3α, 17β-diol (androstanediol) to DHT. Androstanediol is converted to DHT by the 3α-oxidoreductases, 17β-hydroxysteroid dehydrogenase-6 (HSD17B6), retinol dehydrogenase 5 (RDH5), RDH16 and dehydrogenase/reductase family member 9 (DHRS9). The secondary backdoor pathway involves the conversion of DHEA to ASD by HSD3βs, ASD to androstanedione (5α-dione) by SRD5A and 5α-dione is converted to DHT by 3α-oxidoreductases.

Androgen metabolism inhibitors, such as the SRD5A1, 2 or 3 inhibitor, dutasteride, or CYP17A1 inhibitor, abiraterone, inhibit their targets but neither is very effective clinically against CaP. Dutasteride inhibits SRD5A activity, however, intratumoral DHT levels are not depleted. CYP17A1 metabolizes steroids, such as pregnenolone or progesterone, and adrenal androgens, like DHEA, that feed into the 3 androgen metabolism pathways to generate T or DHT. CYP17A1 inhibitors, such as abiraterone decrease intratumoral DHT levels, however, abiraterone was shown to extend survival only approximately 4 months. CaP resistance to abiraterone may be attributed to several mechanisms that include enzyme redundancy, progesterone accumulation that leads to increased CYP17A1 expression or generation of AR splice variants.

Because there are several 3α-oxidoreductases involved in the conversion of androstanediol to DHT, currently no consideration is given to inhibition of these enzymes as a treatment approach.

### SUMMARY OF THE DISCLOSURE

In the present disclosure, we identified that the 3α-oxidoreductases—HSD17B6, RDH16, DHRS9 and RDH5 possess highly conserved catalytic amino acid residues. We further determined that the four 3α-oxidoreductases' catalytic activity is critical for conversion of androsterone (AND) to 5α-dione (5α-dione) and androstanediol to DHT metabolism, and that these 4 enzymes share a common catalytic consensus sequence. Further we also demonstrate that inhibition of the terminal steps of the frontdoor and primary backdoor pathways to DHT synthesis are useful for treatment of advanced CaP. This approach should lower DHT more effectively than inhibitors of 5α-reductases and/or CYP17A1.

In one aspect, this disclosure provides a method of inhibiting the growth of prostate cancer cells comprising contacting the prostate cancer cells with a composition comprising an effective amount of an inhibitor of a catalytic site, which has a consensus sequence that is common to a group of 3α-oxidoreductase enzymes that catalyze the conversion of androstanediol to DHT, wherein the group of 3α-oxidoreductase enzymes comprises 2, 3, 4 or more enzymes. For example, the group of enzymes may comprise HSD17B6, RDH16, DHRS9 and/or RDH5. Thus, the inhibitor can be one that inhibits the catalytic site of one of more of HSD17B6, RDH16, DHRS9 and/or RDH5. The method can further comprise contacting the cells with other known therapeutics for prostate cancer. For example, the method can further comprise contacting the cells with dutasteride and/or abiraterone.

In one aspect, this disclosure provides a method of treating prostate cancer comprising administering to an individual who has prostate cancer a therapeutically effective amount of an inhibitor of a catalytic site, wherein the site is common to (meaning the consensus sequence is found in) a group of 3α-oxidoreductase enzymes that catalyze the conversion of androstanediol to DHT, wherein the group of 3α-oxidoreductase enzymes comprises 2, 3, 4 or more enzymes. For example, the group of enzymes may comprise HSD17B6, RDH16, DHRS9 and/or RDH5. Thus, the inhibitor can be one that inhibits the catalytic site of one of more of HSD17B6, RDH16, DHRS9 and/or RDH5. The method can further comprise administering to the individual other known therapeutics for prostate cancer. For example, the method can further comprise administering to the individual dutasteride and/or abiraterone.

### BRIEF DESCRIPTION OF THE DRAWINGS

FIG. 1: HSD17B6, RDH16, DHRS9 and RDH5 expression in AS-BP, AS-CaP and CRPC. Androgen metabolism pathways for DHT synthesis (A). Diagram of 3α-oxidoreductase activity (B). Consensus sequence of the catalytic site from the four 3α-oxidoreductases (C). IHC of four endogenous 3α-oxidoreductases in AS-BP, AS-CaP and CRPC (D). Positive and negative controls are shown in FIG. 7. Scores showed that HSD17B6 and RDH16 were expressed at higher levels in CRPC than ASBP or AS-CaP tissue (E). Data were presented as the mean +/−SEM. P-values for statistical tests that compared protein expression levels among tissue types and between cytosol and nuclear compartments were listed in Table 4.

FIG. 2: 3α-oxidoreductases were expressed in CaP cell lines and xenografts. qRT-PCR results are shown for 3α-oxidoreductase (A), SRD5A (B), and AR (C) mRNA levels for CaP cell lines and CWR22 and rCWR22 xenografts. Data were presented as mean +/−SEM. P-values for statistical comparisons of gene expression levels among cell lines were listed in Table 5.

FIG. 3: Androgen levels were measured using LC-MS/MS from media and cell pellets of LAPC-4 cells transfected with empty plasmid or expression plasmids encoded with HSD17B6, RDH16, DHRS9 or RDH5. Media and cell pellet androgen levels were combined. Cells were treated for 12 h in SFM alone (A) or SFM with 20 nM DIOL (B) or 1 nM T (C) or 20 nM AND (D). Western blot analysis using DDK antibody was used to confirm enzyme expression (E). Data were presented as mean +/−SEM. P-values generated from comparisons among LAPC-4 cells that expressed 3α-oxidoreductases or LAPC-4 cells with empty plasmid are shown in Table 6. Individual cell pellet and media androgen levels are shown separately in 3 and statistical analysis of the results are shown in Table 7. *p<0.05.

FIG. 4: Mutation of conserved residues impaired 3α-oxidoreductase activity. CV-1 cells were analyzed for 3α-oxidoreductase gene expression (A). 5α-dione levels were measured in CV-1 cells that transiently expressed wild-type HSD17B6, RDH16, DHRS9 or RDH5 wild-type 3α-oxidoreductases catalytic site deletion mutant (Δcat) or double mutant (Y→F, K→R) (B). Transient 3α-oxidoreductase expression was confirmed using western blotting with DDK antibody (C). Data were presented as the mean +/−SEM. P-values for statistical comparisons of media androgen levels between AND or SFM treated CV-1 cells are listed in Table 8. P-values generated from comparisons of media androgen levels among CV-1 cells that expressed wild-type, Y→F, K→R or Δcat 3α-oxidoreductases versus CV-1 cells transfected with empty plasmid are listed in Table 8. *p<0.05.

FIG. 5: 3α-oxidoreductases were expressed in AS-BP and AS-CaP tissues from research subjects treated with finasteride. HSD17B6, RDH16 and RDH5 (A) were detected using IHC in androgen stimulated-benign prostate (AS-BP) and CaP (AS-CaP) tissues. Visual scoring showed 3α-oxidoreductase expression levels did not change with or without finasteride treatment (B). Data were presented as mean +/−SEM. P-values for statistical comparisons of protein expression levels among cytosol and nuclei are listed in Table 9.

FIG. 6: The combination of dutasteride and 3α-oxidoreductase mutants decreased DHT levels greater than dutasteride alone. The effect of dutasteride on DHT levels was determined in LAPC-4 cells that expressed wild-type, Δcat or Y→F, K→R enzymes transiently were treated with or without dutasteride (A). DHT levels were measured in VCaP, C4-2 and CWR-R1 cells transfected with empty plasmid or over-expressing plasmids of wild-type RDH16 or RDH16 Y176F, K180R mutant (B-D). Western blot analysis using DDK antibody confirmed expression of transiently expressed enzymes in LAPC-4, VCaP, C4-2 and CWR-R1 (E-H). The model depicts a coordinated attack on the terminal steps of the front and backdoor androgen metabolism pathways to lower DHT (I). Data were presented as mean +/−SEM. P-values for statistical comparisons of androgen levels between dutasteride and SFM treated cells were listed in Table 10. P-values generated from comparisons of androgen levels among cell lines that expressed wild-type 3α-oxidoreductases, Δcat or Y→F, K→R mutants are listed in S10. *p<0.05.

FIG. 7: IHC positive and negative controls for 3α-oxidoreductase antibodies used to immunostain TMA sections; Related to FIG. 1. (RPCI=Roswell Park Cancer Institute TMA; UNC=University of North Carolina TMA.

FIG. 8: Intracellular T and DHT levels from VCaP cells; Related to FIG. 3. VCaP cells were treated for 12 h in SFM with or without 1 μM Dut, 1 nM T, 20 nM DIOL or 20 nM AND. VCaP cells were harvested, one cell pellet was analyzed for each condition and androgen levels were measured using LC-MS/MS as described in Methods. Dutasteride did not impair DHT synthesis without substrate addition. DHT levels increased when VCaP cells were treated with DIOL or AND and not T. The data suggested VCaP cells use backdoor metabolism to synthesize DHT.

FIG. 9: Intracellular and media androgen levels for LAPC-4 cells transfected with empty plasmid or 3α-oxidoreductases; Related to FIG. 3. Androgen levels were measured using LC-MS/MS from media (A-D) and cell pellets (E-H) of LAPC-4 cells transfected with empty plasmid or over-expressing plasmids of HSD17B6, RDH16, DHRS9 or RDH5. Cells were treated for 12 h in SFM or SFM with 1 nM T, 20 nM DIOL or 20 nM AND. Western blot analysis using DDK antibody was used to confirm enzyme expression (FIG. 3E). Data were presented as mean +/−SEM. P-values generated from comparisons between 3α-oxidoreductases and LAPC-4 cells with empty plasmid are in Table 7.

FIG. 10: Intracellular T levels after treatment with dutasteride or SFM. Related to FIG. 6. LAPC-4, VCaP, C4-2 and CWR-R1 cells were transfected with empty plasmid, wild-type or Y176F,K180R RDH16, treated with SFM or SFM with dutasteride for 12 h and cell pellet androgen levels were measured using LC-MS/MS. T levels were reported for LAPC-4 (A), VCaP (B), C4-2 (C) and CWR-R1 (D)

FIG. 11: Intracellular LNCaP T levels after treatment with dutasteride or SFM. Related to FIG. 6. LNCaP cells were transfected with empty plasmid, wild-type or Y176F, K180R RDH16, treated with SFM or SFM with dutasteride for 12 h and cell pellet androgen levels were measured using LC-MS/MS. T levels for LNCaP (A) and western blot analysis for DDK tag (B).

### DESCRIPTION OF THE DISCLOSURE

The present disclosure is based on the identification of a common catalytic site for the four 3α-oxidoreductases that catalyze the terminal step of the primary backdoor pathway in the conversion of androstanediol to DHT. This disclosure provides methods for the treatment of prostate cancer comprising administering to an individual a therapeutically effective amount of an agent that inhibits one or more of the 3α-oxidoreductases. For example, the agent may inhibit 1, 2, 3, or 4 or more of the known 3α-oxidoreductases. The 3α-oxidoreductases can be HSD17B6, RDH16, DHRS9 and/or RDH5, and/or others.

This disclosure provides a method of inhibiting the growth of a prostate cancer cell comprising the step of inhibiting the activity of one or more, or two or more 3α-oxidoreductases. The 3α-oxidoreductases can be HSD17B6, RDH16, DHRS9 and/or RDH5, and/or others. The activity of one or more, or two or more 3α-oxidoreductases may be inhibited by inhibiting the activity of the enzyme (protein) or inhibiting at the transcription/translation level. For example, specific small molecule inhibitors, including peptides, and/or inhibitory RNAs may be used. Methods of inhibiting the enzyme activity also includes targeted inhibition, such as site-directed mutagenesis or gene editing techniques (such as clustered regularly interspaced short palindromic repeats or CRISPR). For example, methods can be based on interfering with or altering the common amino acids in the consensus sequence GGYX₁X₂SK (SEQ ID NO: 1). For example, the catalytic activity of this site may be interfered with, or the Y and the K may be changed to another amino acid.

This disclosure provides a method of reducing the conversion of androstanediol to DHT in prostate cells comprising inhibiting the activity of a more than one 3α-oxidoreductases. The enzymes can be two or more of HSD17B6, RDH16, DHRS9 and RDH5.

This disclosure provides a method of treating prostate cancer comprising administering to an individual who has prostate cancer a therapeutically effective amount of an inhibitor of a catalytic site common to a group of 3α-oxidoreductase enzymes that catalyze the conversion of androstanediol to DHT, wherein the group of 3α-oxidoreductase enzymes comprises 2, 3, 4 or more 3α-oxidoreductase enzymes. In one embodiment, the enzymes are at least: HSD17B6, RDH16, DHRS9 and/or RDH5. The method may further comprise administering to the individual a composition comprising another therapeutic effective against prostate cancer. For example, the method may comprise administering to the individual a composition comprising dutasteride and/or abiraterone, which may be administered concurrently or sequentially with the 3α-oxidoreductase enzymes inhibitor.

This disclosure provides a method of inhibition of growth of prostate cancer cells comprising contacting the cells with an effective amount of an inhibitor of a catalytic site common to a group of 3α-oxidoreductase enzymes that catalyze the conversion of androstanediol to DHT. The group of 3α-oxidoreductase enzymes that catalyze the conversion of androstanediol to DHT and which have the consensus catalytic site sequence can comprise 2, 3, 4 or more 3α-oxidoreductase enzymes. In one embodiment, the enzymes are HSD17B6, RDH16, DHRS9 and/or RDH5. The method may further comprise contacting the cells with a composition comprising another inhibitor of growth of prostate cancer cells. For example, the method may comprise contacting the cells with a composition comprising dutasteride and/or abiraterone. The cells may be contacted concurrently or sequentially with the 3α-oxidoreductase enzymes inhibitor.

The activity of two or more 3α-oxidoreductase enzymes can be inhibited by contact with a composition comprising a single inhibitor of the consensus catalytic sequence, which can inhibit the activity of more than one 3α-oxidoreductase enzymes, and preferably of all the 3α-oxidoreductases. The composition can comprise other inhibitors of growth of cancer cells such as dutasteride and/or abiraterone.

The disclosure provides compositions for use in the inhibition of growth or prostate cancer cells or treatment of prostate cancer. The compositions comprise inhibitors of two or more 3α-oxidoreductase enzymes which comprise a sequence encoding a catalytic site that is common to the two or more 3α-oxidoreductase enzymes. For example, the inhibitors may inhibit one or more of HSD17B6, RDH16, DHRS9 and RDH5. The composition may further comprise another inhibitor of growth of prostate cancer cells, such as dutasteride and/or abiraterone.

By referring to 3α-oxidoreductase enzymes having a common catalytic site is meant that their amino acid sequence comprises a consensus sequence for the catalytic site. The consensus sequence can be GGYX₁X₂SK (SEQ ID NO: 1), wherein X₁X₂ can be any amino acids. For example, X₁ may be C or T, and X₂ may be V, I or P. These amino acids are found in positions 174-180 of the amino acid sequences of the HSD17B6, RDH16, DHRS9 and RDH5 3α-oxidoreductase enzymes (Accession Nos. NP 003716.2, NP 003699.3, NP 002896.2, and NP 954674.1 respectively).

The disclosure provides prostate cancer cells in which inhibitors of two or more 3α-oxidoreductase enzymes have been introduced, which inhibitors may be inhibitors of the two or more 3α-oxidoreductase enzyme protein or mRNAs. In one embodiment, the two or more 3α-oxidoreductase enzymes are HSD17B6, RDH16, DHRS9 and RDH5.

The disclosure also provides prostate cells in which one or more of the following enzymes have been inactivated, or are non-functional or minimally functional: HSD17B6, RDH16, DHRS9 and RDH5. The enzymes may have been inactivated, or rendered non-functional or minimally functional by inactivating at the protein level or at the DNA/RNA level.

The term “therapeutically effective amount” as used herein refers to an amount of an agent sufficient to achieve, in a single or multiple doses, the intended purpose of treatment. For example, an effective amount to treat prostate cancer is an amount sufficient to kill prostate cancer cells. The exact amount desired or required will vary depending on the particular compound or composition used, its mode of administration and the like. Appropriate effective amount can be determined by one of ordinary skill in the art informed by the instant disclosure using only routine experimentation.

Within the meaning of the disclosure, “treatment” also includes relapse, or prophylaxis as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy.

An agent for inhibiting or reducing the activity of one or more 3α-oxidoreductases may act by inhibiting the nucleic acids or by inhibiting the protein. For example, the agent may act at the mRNA level or at the protein level. Inhibition of the 3α-oxidoreductases can comprise pharmacological inhibition of their activity. In certain approaches, pharmacologic inhibition comprises use of enzyme-specific small molecules for enzymatic activity inhibition, or neutralizing antibodies, or other enzyme-specific biologics.

In one aspect, the disclosure includes interfering with the activity of mRNA encoding one or more of the 3α-oxidoreductases, and/or inhibiting the transcription or translation of the mRNA, and as a result reducing expression of the enzyme(s). Reducing mRNA can involve introducing into cells that express the enzyme a molecule such as a polynucleotide that can inhibit translation of enzyme-encoding mRNA, and/or can participate in and/or facilitate RNAi-mediated reduction of the mRNA. For example, an antisense polynucleotide can be used to inhibit translation of the mRNA. Antisense nucleic acids can be DNA or RNA molecules that are complementary to at least a portion of the targeted mRNA. For example, the DNA or RNA molecules may be complementary to the portion of the mRNA that encodes for the common catalytic region of the 3α-oxidoreductases. The DNA or RNA molecules may be from 5 to 15 nucleotides. The polynucleotides for use in targeting mRNA may be modified, such as, for example, to be resistant to nucleases.

As an example, this disclosure includes RNAi-mediated reduction in mRNA. RNAi-based inhibition can be achieved using any suitable RNA polynucleotide that is targeted to an enzyme-mRNA. For example, a single stranded or double stranded RNA, wherein at least one strand is complementary to the targeted mRNA, can be introduced into the cell to promote RNAi-based degradation of target mRNA. MicroRNA (miRNA) targeted to the mRNA can be used. A ribozyme that can specifically cleave target mRNA can be used. Small interfering RNA (siRNA) can be used. siRNA (or ribozymes) can be introduced directly, for example, as a double stranded siRNA complex, or by using a modified expression vector, such as a lentiviral vector, to produce an shRNA. As is known in the art, shRNAs adopt a typical hairpin secondary structure that contains a paired sense and antisense portion, and a short loop sequence between the paired sense and antisense portions. shRNA is delivered to the cytoplasm where it is processed by DICER into siRNAs. siRNA is recognized by RNA-induced silencing complex (RISC), and once incorporated into RISC, siRNAs facilitate cleavage and degradation of targeted mRNA. A shRNA polynucleotide used to suppress mRNA expression can comprise or consist of between 45-100 nucleotides, inclusive, and including all integers between 45 and 100, and all ranges there between. As an example, the portion of the shRNA that is complementary to the target mRNA can be from 21-29 nucleotides, inclusive, and including all integers between 21 and 29.

For delivering siRNA via shRNA, modified lentiviral vectors can be made and used according to standard techniques, given the benefit of the present disclosure. In certain approaches, modified lentiviruses are used to stably infect target cells, and may integrate into a chromosome in the targeted cells. For example, see Titus M A, Zeithaml B, Kantor B, Li X, Haack K, Moore D T, Wilson E M, Mohler J L, Kafri T. Dominant-negative androgen receptor inhibition of intracrine androgen-dependent growth of castration-recurrent prostate cancer. PLoS One 2012;7(1): e30192.

Other compositions to inhibit one or more enzymes may be used in the form of pharmaceutical compositions. The pharmaceutical composition of the invention may be administered by any route that is appropriate, including but not limited to parenteral or oral administration. The pharmaceutical compositions for parenteral administration include solutions, suspensions, emulsions, and solid injectable compositions that are dissolved or suspended in a solvent before use. The injections may be prepared by dissolving, suspending or emulsifying one or more of the active ingredients in a diluent. Examples of diluents are distilled water for injection, physiological saline, vegetable oil, alcohol, and a combination thereof. Further, the injections may contain stabilizers, solubilizers, suspending agents, emulsifiers, soothing agents, buffers, preservatives, etc. The injections, are sterilized in the final formulation step or prepared by sterile procedure. The pharmaceutical composition of the invention may also be formulated into a sterile solid preparation, for example, by freeze-drying, and may be used after sterilized or dissolved in sterile injectable water or other sterile diluent(s) immediately before use. The compositions described can include one or more standard pharmaceutically acceptable carriers. Some examples herein of pharmaceutically acceptable carriers can be found in: Remington: The Science and Practice of Pharmacy (2005) 21st Edition, Philadelphia, Pa. Lippincott Williams & Wilkins.

The method of the present disclosure may be carried out in an individual who has been diagnosed with prostate cancer (i.e., therapeutic use). It may also be carried out in individuals who have a relapse or a high risk of relapse after being treated for CaP.

Inhibitors of the catalytic site of 3α-oxidoreductases may be used alone, with other agents with similar effects, or with other modalities, including chemotherapeutic agents, surgery, radiation and the like. For example, inhibitors of the common catalytic site of 3α-oxidoreductases may be used with dutasteride and/or abiraterone. Currently, no clinical inhibitors against these 3α-oxidoreductases are available. Therefore, an inhibitor that blocks the 3α-oxidoreductases may be used in combination with existing therapies like dutasteride to provide a new treatment strategy to block the key enzymatic steps of the frontdoor, primary and secondary backdoor pathways, which may decrease DHT levels better than ADT alone.

Further reduction of tissue DHT levels by inhibiting the last step(s) in intracrine metabolism may improve response to ADT or induce re-remission of CRCP and improve survival of men with advanced CaP.

This disclosure provides a method of treating prostate cancer comprising administering to an individual who has prostate cancer a therapeutically effective amount of an inhibitor of a catalytic site common to a group of 3α-oxidoreductase enzymes that catalyze the conversion of androstanediol to DHT, wherein the group of 3α-oxidoreductase enzymes comprises 2, 3, 4 or more enzymes. The enzymes may be at least: HSD17B6, RDH16, DHRS9 and/or RDH5. The method can further comprise administering to the individual a composition comprising a therapeutic effective against prostate cancer, such as dutasteride and/or abiraterone.

The present disclosure provides a method for identifying agents that can synergistically, with other anti-prostate cancer agents (such as, for example, dutasteride and/or abiraterone) inhibit the activity of 3α-oxidoreductase enzymes. For example, the agents may be identified by contacting prostate cancer cells with candidate agents to determine if one or more of the 3α-oxidoreductase enzymes are synergistically inhibited upon further contact with dutasteride and/or abiraterone.

The following examples further describe the disclosure. These examples are intended to be illustrative and not limiting in any way.

### EXAMPLE 1

This example: i) demonstrates that the 3α-oxidoreductases are expressed in AS-CaP and CRPC, ii) identifies the catalytic residues necessary to catalyze the terminal step of the primary backdoor pathway, androstanediol to DHT, and iii) provides evidence that combined SRD5A and 3α-oxidoreductase inhibition lowers DHT levels further than inhibition of either enzyme family alone.

### Experimental Procedures

### Cell culture

Human CaP lines LAPC-4 (Klein et al., 1997, Nat Med 3, 402-408), LNCaP-RPCI (LNCaP) (Horoszewicz et al., 1983, Cancer Res 43, 1809-1818), PC-3 (ATCC, Manassas, Va.) and LNCaP-C4-2 (C4-2) cells (Thalmann et al., 1994; Wu et al., 1994, Cancer Res 54, 2577-2581) were cultured in RPMI 1640 (Mediatech, Inc., Manassas, Va.). CWR-R1 cells (Gregory et al., 2001, Cancer Res 61, 2892-2898) was cultured using Richter's Improved media (Corning). CV-1 monkey kidney cells, DU145 (Mickey et al., 1980, Prog Clin Biol Res 37, 67-84) and VCaP cells (ATCC) were cultured in DMEM (Corning, Corning, N.Y.). RPMI and DMEM media were supplemented with 10% fetal bovine serum (FBS, Corning) and 2 mM glutamine (Corning). CWR-R1 cells were cultured in Richter's Improved Media (Corning) supplemented with 1% epidermal growth factor (Thermo Fisher Scientific, Waltham, Mass.), 1% insulin-transferrin-sodium selenite supplement (Roche, Indianapolis, Ind.), 1% nicotinamide (Calbiochem, Billerica, Mass.) and 2% FBS. Androgen-dependent CWR22 (Wainstein et al., 1994, Cancer Res 54, 6049-6052) and castration-recurrent CWR22 (rCWR22) (Nagabhushan et al., 1996, Cancer Res 56, 3042-3046) human CaP xenografts were propagated in immunocompromised nude mice.

All cell lines and xenografts were authenticated using genomic profiling in the Genomic Shared Resource. DNA profiles were acquired using 15 short tandem repeat (STR) loci and an amelogenin gender-specific marker. Test and control samples were amplified using the AmpFLSTR® Identifiler® Plus PCR Amplification Kit (Thermo Fisher Scientific, Waltham, Mass.) using the Verti 96-well Thermal Cycler (Applied Biosystems, Foster City, Calif.) in 9600 Emulation Mode (initial denature: 95° C. 11 min, 28 cycles of denature: 94° C. 20 sec and anneal/extend: 59° C. 3 min, final extension: 60° C. 10 min and hold: 12° C.). PCR products were evaluated using the 3130xl Genetic Analyzer (Applied Biosystems) and analyzed using GeneMapper v4.0 (Applied Biosystems). Eight of the 15 STRs and amelogenin from the DNA profile for the cell lines were compared to the ATCC STR database (atcc.org/STR%20Database.aspx?slp=1) and the DSMZ combined Online STR Matching Analysis (dsmz.de/fp/cgi-bin/str.html). All matches above 80% were considered the same lineage.

LAPC-4 cells were plated at 1.2×10⁵/well and CV-1 cells at 1×10⁴/well in 6-well tissue culture plates (Corning). Media and cell pellets from two 6-well plates were combined to generate 1 media and 1 cell pellet sample for liquid chromatography-tandem mass spectrometry (LC-MS/MS) analysis. LAPC-4 cells were transfected using the Effectene Transfection Kit (Invitrogen, Grand Island, N.Y.). Forty-eight h after transfection, growth media was removed and LAPC-4 cells were washed once with Dulbecco's phosphate buffered saline (PBS, Corning). LAPC-4 cells were treated with serum-free complete media (SFM, Corning) alone or with 1 nM T, 20 nM DIOL 20 nM 5α-androstan-3α-o1-17-one (androsterone; AND) (Steraloids, Newport, R.I.) or 1 μM dutasteride (Selleckchem, Houston, Tex.) for 12 h. CV-1 cells were transfected using X-tremeGene HP transfection reagent (Roche Diagnostics Corporation, Indianapolis, Ind.). After 48 h, growth media were aspirated, CV-1 cells were washed once with PBS and incubated in SFM alone or with 20 nM AND (Steraloids) for 12 h.

After 12 h treatment, media (total 24 mL) were collected in 50 mL conical tubes (Corning). Cells were released using trypsin and collected in 15 mL conical tubes (Corning). The cells were washed 3 times using PBS, re-suspended in 1 mL PBS and 5% of the cell suspension was removed for protein concentration measurement and western blot analysis. The remaining 95% of the cell suspension was centrifuged and the supernatant was removed and discarded. Cell pellets were stored at −80° C. until analyzed using LC-MS/MS.

### Constraint-based Multiple Protein Alignment Tool (COBALT)

The COBALT protein sequence alignment tool was accessed using the National Center for Biotechnology (NCBI) website (ncbi.nlm.nih.gov/tools/cobalt/cobalt.cgi). Accession numbers for the 3α-oxidoreductases, HSD17B6, RDH16, DHRS9 and RDH5, were acquired using the NCBI protein database (ncbi.nlm.nih.gov/protein) and UniProtKB (uniprot.org/). Amino acid sequences of the four 3α-oxidoreductases were analyzed using COBALT and compared to other 3α-oxidoreductases and the SRD5A and CYP17 families to determine whether the catalytic site was conserved and specific to HSD17B6, RDH16, DHRS9 and RDH5.

### Site-Directed Mutagenesis

Site-directed mutagenesis was performed using the QuikChange® Lightning Site-Directed Mutagenesis Kit (Strategene, Foster City, Calif.), using Strategene's protocol. pCMV6-entry expression plasmids with C-terminal MYC-DDK tag encoded with HSD17B6, RDH16, DHRS9 or RDH5 were purchased from Origene (Origene, Rockville, Md.). 3α-oxidoreductase primers (Integrated DNA Technologies, Coralville, Iowa) were used to delete the catalytic site (Δcat) or generate double mutations (Y→F, K→R) (Table 1). Plasmids were purified using PureYield Plasmid Miniprep System (Promega, Madison, Wis.) and sequenced at the Roswell Park Cancer Institute Genomic Shared Resource. Polymerase chain reaction (PCR) for plasmid sequencing was performed using plasmid templates and Big Dye Terminator v3.1 Master Mix Kit (Life Technologies, Carlsbad, Calif.). PCR products were purified using Sephadex-G50 (Sigma-Aldrich, St. Louis, Mo.) into multiscreen HV plates (Thermo Fisher Scientific). Eluted samples were analyzed using 3130xl ABI Prism Genetic Analyzer. Sequence data were analyzed using Sequencing Analysis 5.2 software (Life Technologies).

TABLE 1
Primer list

| Site directed mutagenesis primers; | | | | SEQ |
|---|---|---|---|---|
| Related to site-directed mutagenesis methods | | | | ID NO |
| HSD17B6 | Δcat | FW | 5′-gggaagagttgctttctttgtatatggagtggaagccttttc-3′ | 5 |
| | | RV | 5′-gaaaaggcttccactccatatacaaagaaagcaactcttccc-3′ | 6 |
| | Y176F, K180R | FW | 5′-ggcttctgtgtctccaggtatggagtggaagcc-3′ | 7 |
| | | RV | 5′-ggcttccactccatacctggagacacagaagcc-3′ | 8 |
| RDH16 | Δcat | FW | 5′-gggtgtcactttttggttatggcgtggaagcctt-3′ | 9 |
| | | RV | 5′-aaggcttccacgccataaccaaaaagtgacaccc-3′ | 10 |
| | Y176F, K180R | FW | 5′-gcttctgcatctccaggtatggcgtggaagc-3′ | 11 |
| | | RV | 5′-gcttccacgccatacctggagatgcagaagc-3′ | 12 |
| DHRS9 | Δcat | FW | 5′-cgccttgcaatcgttggatatgcagtggaaggtttc-3′ | 13 |
| | | RV | 5′-gaaaccttccactgcatatccaacgattgcaaggcg-3′ | 14 |
| | Y176F, K180R | FW | 5′-aaggcttccacgccataaccaaaaagtgacaccc-3′ | 15 |
| | | RV | 5′-aaccttccactgcatatctggatggagtaaagccc-3′ | 16 |
| RDH5 | Δcat | FW | 5′-ctggcagccaatggttttggcctggaggcc-3′ | 17 |
| | | RV | 5′-ggcctccaggccaaaaccattggctgccag-3′ | 18 |
| | Y175F, K179R | FW | 5′-gcttctgtgtctccagatttggcctggaggc-3′ | 19 |
| | | RV | 5′-gcctccaggccaaatctggagacacagaagc-3′ | 20 |

Catalytic deletion (Δcat)

| Primers used for qRT-PCR; Related to qRT-PCR methods | | | | SEQ |
|---|---|---|---|---|
| Gene | | Primer sequences | | ID NO |
| 1 | HSD17B6 | FW | 5′-AGC ATG CTT CCT TTG GTG AGG AGA-3′ | 21 |
| | | RV | 5′-TTC CCG TTC TGA AGT AGC CAG GTT-3′ | 22 |
| 2 | RDH16 | FW | 5′-TGT GGT CAA CGT CTC CAG TGT CAT-3′ | 23 |
| | | RV | 5′-AGA GAA GGC TTC CAC GCC ATA CTT-3′ | 24 |
| 3 | DHRS9 | FW | 5′-GTC AAG AAA GCT CAA GGG AGA G-3′ | 25 |
| | | RV | 5′-CCA CTG CAT ATT TGG ATG GAG TA-3′ | 26 |
| 4 | RDH5 | FW | 5′-GGC GGG ATG TAG CTC ATT T-3′ | 27 |
| | | RV | 5′-TTC TCC AGA CTC TCC AGG TT-3′ | 28 |
| 5 | SRD5A1 | FW | 5′-TTC TGT ACC TGT AAC GGC TAT TT-3′ | 29 |
| | | RV | 5′-GGG ATC TGT TAC CCA GTC ATC-3′ | 30 |
| 6 | SRD5A2 | FW | 5′-CCT TCT GCA CTG GAA ATG GA-3′ | 31 |
| | | RV | 5′-CAC CCA AGC TAA ACC GTA TGT-3′ | 32 |
| 7 | SRD5A3 | FW | 5′-GGT CAT CTG CCC ATC AGT ATA AG-3′ | 33 |
| | | RV | 5′-CCA AAT GGG ATC CTG TGG TTA-3′ | 34 |
| 8 | AR | FW | 5′-CGG CTA ATG GGT GGA ATC TAA-3′ | 35 |
| | (N-term) | RV | 5′-GGT TAC ACC AAA GGG CTA GAA-3′ | 36 |
| 9 | B2M | FW | 5′-GAC TTG TAG AGA GAC AGG GTA GA-3′ | 37 |
| | | RV | 5′-TAG GAG GGC TGG CAA CTT AG-3′ | 38 |

### Quantitative Real-Time Polymerase Chain Reaction (qRT-PCR)

RNA extraction was performed using the RNeasy Plus Mini Kit (Qiagen, Valencia, Calif.). Samples generated from 9 cell lines were plated at 1×10⁶ cells/T25 cell culture flask (Corning). Cells were harvested using 0.05% trypsin and washed 3 times using PBS. PBS was removed and RLT lysis buffer was added to lyse the cell pellets. Frozen tissues from 2 xenografts (CWR22 and rCWR22) were Dounce homogenized in RLT lysis buffer. Lysates were passed through QIAshredder columns and RNA was extracted using RNeasy spin columns. Genomic DNA contamination was assessed using PCR and intron spanning GAPDH primers. Genomic DNA contamination was removed using a DNA-free DNA Removal Kit (Life Technologies). RNA was analyzed using PCR after DNase treatment to confirm genomic DNA was removed.

First strand complementary DNA (cDNA) was generated using 2 μg RNA and the High-Capacity cDNA Reverse Transcription Kit (10× RT Buffer, 10× Random Primers, 25× 100 mM deoxyNTP mix and 50 U/μL MultiScribe-Reverse Transcriptase; Applied Biosystems) and RNAse inhibitor in 10 μL reactions. The Primer Quest Primer Design Tool was used to design qRT-PCR primers (Integrated DNA Technologies) (Table 1).

qRT-PCR reactions included 12.μL of SYBR Green PCR Master Mix (Applied Biosystems), 0.1 μL of 10 mM forward and reverse primers, 2.5 μL of 100 ng/μL cDNA (250 ng final concentration) and 9.8 μL distilled deionized (dd-H₂O) for a final reaction volume 25 μL. Reactions were performed in 96-well plates. Gene expression was analyzed using 7300 Real Time System (Applied Biosystems). The qRT-PCR reaction parameters were 95° C. for 30 sec, 60° C. for 30 sec repeated 39 times, 95° C. for 5 sec and melt curve 65° C.-95° C. All procedures were performed with 3 technical replicates and 3 biological replicates. Cycle threshold (Ct) values were normalized against β2 microglobulin (B2M) that was selected based on unchanged B2M expression in SFM (data not shown). Ct values for negative RT controls and no template controls were reported as undefined. Relative gene abundance was calculated using 2\hat{ }-(normalized Ct).

### LC-MS/MS

Cell pellet and media samples were analyzed over 9 runs for 5 androgens T, DHT, dehydroepiandrosterone, ASD and AND using a validated LC-MS/MS method. 5α-dione was also measured using this assay and data were included although results did not pass the strict validation acceptance criteria used for other androgens. Study samples were quantitated using aqueous-based spiked calibration standards and pre-spiked quality control samples prepared in 2× charcoal-stripped human postmenopausal female plasma (Bioreclamation, LLC, Westbury, N.Y.). Performance data for calibrators, quality controls and calibration ranges were listed in Table 2. Values below the lower limit of quantitation were treated as zero.

TABLE 2
Related to LC-MS/MS methods

| | Calibrator Accuracy (%) | | Calibrator Precision (%) | |
|---|---|---|---|---|
| Compound | Mean | Range | Mean | Range |
| ASD | 100 | 92.7-104 | 2.50 | 1.09-4.14 |
| T | 100 | 92.1-104 | 2.22 | 0.866-3.41 |
| DHEA | 100 | 98.5-101 | 4.20 | 2.75-7.79 |
| DHT | 100 | 92.5-104 | 2.89 | 1.18-5.11 |
| AND | 100 | 93.9-105 | 3.80 | 1.94-4.83 |
| 5α-dione | 100 | 99.4-103 | 3.68 | 2.96-4.55 |

| | |
|---|---|
| QC Accuracy (%) | QC Precision (%) |

| Compound | Mean | Range | Mean | Range |
|---|---|---|---|---|
| ASD | 95.9 | 93.2-97.2 | 7.17 | 4.96-10.9 |
| T | 97.7 | 95.1-100 | 6.28 | 5.63-7.10 |
| DHEA | 96.8 | 95.6-98.2 | 7.67 | 5.79-9.06 |
| DHT | 101 | 96.9-103 | 7.63 | 6.26-8.72 |
| AND | 98.1 | 93.4-101 | 7.15 | 6.27-7.90 |
| 5α-dione | 93.4 | 90.7-94.8 | 15.9 | 14.9-17.2 |

| | | |
|---|---|---|
| Compound | Serum calibration ranges | LLOQ |

| | | | | |
|---|---|---|---|---|
| ASD | 0.00625-3.75 | ng/mL | 0.00625 | ng/mL |
| T | 0.00625-3.75 | ng/mL | 0.00625 | ng/mL |
| DHEA | 0.200-7.50 | ng/mL | 0.200 | ng/mL |
| DHT | 0.0125-7.50 | ng/mL | 0.0125 | ng/mL |
| AND | 0.200-7.50 | ng/mL | 0.200 | ng/mL |
| 5α-dione | 0.200-7.50 | ng/mL | 0.200 | ng/mL |

Table 2. Related to LC-MS/MS methods
5α-dione did not pass standard acceptance criteria during assay validation (i.e., theoretical concentration ± 15% as recommended by FDA's Bioanalytical Guidance), but 5α-dione levels were reported since 5α-dione is an integral component of the androgen pathway under study. Overall performance statistics of the calibrators and quality controls for the nine analytical runs disclosed that six runs passed the normal ±15% criteria, one run passed at ±20%, one run passed at ±25% and one run passed at ±30%.

Androgen concentrations (pmoles/mg protein) in cell lysates measured using LC-MS/MS (ng/mL) were multiplied by the total volume of the lysate (1 mL), normalized by the total amount of protein of the cell pellet (mg), divided by the molecular weight of the androgen (ng/nmole), and converted to pmoles. Media androgen concentrations (ng/mL) were multiplied by the total volume of media (24 mL), normalized against total protein of the cell culture (mg), divided by the molecular weight of the androgen (ng/nmole) and converted to pmoles. Cell pellet and media androgen concentrations were reported combined in Results and separately in Supplemental Results. Experiments were performed in triplicate.

### Western Blotting

Cells removed from −80° C. storage were resuspended in ubiquitin extraction lysis buffer (150 mM NaCl, 50 mM Tris-HCl, pH 7.4, 5 mM EDTA, 1% NP40, 0.5% sodium deoxycholate (all from Fisher, Pittsburg, Pa.)) and 0.1% SDS (Quality Biological, Gathersburg, Md.). Halt Protease Inhibitor Cocktail (Sigma) was added to the ubiquitin lysis buffer just before cells were lysed. Cells were freeze-thawed three times and centrifuged at 14,000× g for 15 min. Supernatants were transferred to clean microfuge tubes. Protein was quantified using the Protein Determination Kit (BioRad) and analyzed in flat bottom 96-well plates using an EL800 University Microplate Reader (BioTek Instruments) and KC Junior software (Bio-Tek Instruments). SDS-polyacrylamide gel electrophoresis (PAGE) was performed using 4-15% Mini Trans-Blot cell (BioRad). Protein was transferred to Immuno-Blot PVDF membranes for Protein Blot (BioRad) and blocked in 5% milk in Tris-buffered saline with Tween 20 (TBST, Amersham Bioscience, GE Healthcare Bio-Sciences, Pittsburg, Pa.) for 30 min.

Membranes were incubated with DDK targeted antibody (Origene) 1:1000 overnight at room temperature. After incubation, blots were washed three times with TBST (Amersham Bioscience) for 10 min each. Washed blots were incubated with goat anti-mouse secondary antibody (Jackson ImmunoResearch Laboratories, West Grove, Pa.) 1:1000 for 1 h at room temperature. Blots were washed with TBST three times for 10 min each and protein expression was measured using the Pierce ECL Western Blotting Substrate (Life Technologies). Immunoblots were washed with TBST, blocked in 5% milk in TBST, reprobed for tubulin (1:1000 for 1 h at room temperature; Abcam, Cambridge, Mass.) and incubated with goat anti-rabbit secondary antibody (1:1000 for 1 h at room temperature; Jackson ImmunoResearch Laboratories).

### Tissue Microarray (TMA) Construction

Matched androgen-stimulated benign prostate (AS-BP) and androgen-stimulated CaP (AS-CaP) tissue specimens were collected from 36 patients who underwent radical prostatectomy. CRPC specimens were collected from 36 patients who underwent transurethral resection of the prostate for urinary retention from CaP that recurred during ADT. Specimens were collected between 1991 and 2011 at the Roswell Park Cancer Institute or the University of North Carolina (Chapel Hill, N.C.). TMAs were constructed (0.6 millimeter tissue cores) from formalin-fixed, paraffin-embedded donor blocks from each patient, which was guided by RPCI or UNC genitourinary pathologists. TMAs contained control tissue from lung, tonsil, liver, kidney, colon, spleen, cervix, thyroid, ovary, testis, myometrium and brain.

TMAs were constructed using the same process on tissues collected from a randomized, double-blind, placebo-controlled clinical trial of selenium supplementation and finasteride treatment of patients with CaP prior to robotic prostatectomy (Selenium and Finasteride Pre-Treatment Trial ID:NCT00736645). Forty-seven patients scheduled for radical prostatectomy were randomized into one of four treatment groups: placebo, finasteride, selenium or combination finasteride and selenium.

### Immunohistochemistry (IHC)

TMA sections were de-paraffinized, rehydrated under an alcohol gradient and antigen retrieved using Reveal Decloaker (Biocare Medical, Concord, Calif.) for 30 min at 110° C. and 5.5-6.0 psi. Sections were immunostained for the four 3α-oxidoreductases and AR as described (Table 3). Enzymatic activity was assayed using 3,3′-Diaminobenzidine (Sigma-Aldrich) and sections were counterstained with hematoxylin (Vector Laboratories, Burlingame, Calif.). Sections were dehydrated and mounted using permanent mounting medium. Section images were collected using a Leica DFC0425C camera mounted on a Leica DMRA2 microscope (Leica Microsystems Inc., Buffalo Grove, Ill.). Protein expression was determined by three scorers who assessed the immunostain intensity and assigned values between zero (no immunostain) and three (dark immunostain) for 100 cells per core that generated a final score between zero and 300.

TABLE 3
IHC antibodies and methods; Related to IHC methods

| Target | HSD17B6 | RDH16 | DHRS9 | RDH5 | AR |
|---|---|---|---|---|---|
| Block step | None | Normal | Back- | None | None |
| | | goat | ground | | |
| | | serum | Punisher | | |
| Primary | HSD17B6 | RDH16 | DHRS9 | RDH5 | AR |
| antibody | | | | | |
| Source | Abcam | Abcam | Abcam | Everest | DAKO |
| Primary ab | Ab88892 | Ab89653 | Ab89698 | EB10078 | M3562 |
| catalog # | | | | | |
| Host | Mouse | Rabbit | Rabbit | Goat | Mouse |
| Primary ab | 1:100 | 1:200 | 1:600 | 1:100 | 1:100 |
| dilution | | | | | |
| Secondary | Goat | Goat | Goat | Rabbit | Goat |
| ab | anti- | anti- | anti- | anti- | anti- |
| | mouse | rabbit | rabbit | goat | mouse |
| Source | DAKO | DAKO | MACH 4 | DAKO | DAKO |
| | | | HRP | | |
| | | | polymer | | |
| Secondary | P0447 | P0448 | MRH534H | P0160 | P0447 |
| ab catalog # | | | | | |
| Secondary | 1:100 | 1:100 | 3-4 drops | 1:100 | 1:100 |
| ab dilution | | | | | |

### Statistical Analysis

IHC and qRT-PCR expression data were modeled as a function of tissue type (AS-BP, AS-CaP or CRPC) or cell line (VCaP, LNCaP, LAPC-4, C4-2, PC-3, DU145, CWR-R1, CWR22 or rCWR22). Androgen concentrations were measured using LC-MS/MS and modeled for cell pellets, media and both as a function of enzyme (control, HSD17B6, RDH16, DHRS9 or RDH5), treatment (SFM, T, DIOL, AND or dutasteride), expressed wild-type, Δcat or Y→F, K→R mutant enzymes, interaction terms and random (replicate or subject) effects using a linear mixed model. The factors, factor levels, interaction terms and random effects included in each model depended on the specific experiment and research question addressed. Mean differences were evaluated using Dunnett or Tukey-Kramer adjusted F-tests about the appropriate linear contrasts of model estimates. All model assumptions were verified graphically using quantile-quantile and residual plots, with transformations applied when appropriate. All analyses were conducted in SAS v9.4 (Cary, NC) at a nominal significance level of 0.05.

### Additional Methods

Cell pellets were resuspended in HPLC-grade water and vortexed or sonicated to disrupt cell membranes to obtain a homogeneous suspension. Samples were extracted using 0.25 ml quality control, plasma blank or sample in 0.75-2.0 mL HPLC-grade water, 0.1 ml internal standard (IS) solution (75.0/225 pg/mL d₃-T/d₃-DHT) and 4 mL methyl-tert-butyl ether (MTBE, Omnisolve®, EMD Milipore, Billerica, Mass.) in glass screw-top tubes. Calibrators were prepared using 50 μl spiking solution prepared in 75% methanol and added to the extraction tube. Either the entire 1 mL cell pellet suspension was added to the extraction tube and the original container rinsed with 25% methanol in water or samples were volume and compositionally corrected. Tubes were capped with Teflon-lined caps, vortexed, rotated 15 min and centrifuged using a Sorvall model RT6000B centrifuge (Thermo Scientific) at 2,800 rpm and 4° C. for 15-30 min to separate liquid phases. The aqueous phase was frozen in a dry ice/acetone bath and MTBE layer was poured into a clean glass conical tube. MTBE was evaporated at 37° C. with nitrogen and the residue was reconstituted with 60% methanol. The suspension was centrifuged using Heraeus Multifuge X3R centrifuge (Thermo Scientific) at 2,800 rpm and 4° C. for 5 min to separate insoluble materials. An aliquot of the supernatant was injected.

LC-MS/MS analysis of the extracted samples was performed using a Prominence UFLC System (Shimadzu Scientific Instruments, Kyoto, Japan) a QTRAP® 5500 mass spectrometer (AB Sciex, Framingham, Mass.) with an electrospray ionization source and two 10-port switching valves (Model EPC10W Valco Instruments Co. Inc., Houston, Tex.). The first switching valve was mounted in the column oven and was used to perform inline sample cleanup. The second valve functioned as a divert valve to switch the column eluent between waste and the mass spectrometer. Chromatographic separation was achieved using a Phenomenex® Luna® C18(2) column (part number 00F-4251-B0) preceded by a Phenomenex® SecurityGuard™ cartridge (C18, part number AJ0-4286). The HPLC column was maintained at 60° C. and flow rate was 175 μL/min using a biphasic gradient. Mobile phase A was 65% methanol containing 400 μL 1 M ammonium formate and 65 μL concentrated formic acid per liter. Mobile phase B was 100% methanol containing 400 μL 1 M ammonium formate and 65 μL concentrated formic acid per liter.

Analytes were detected using multiple reaction monitoring in positive ion mode controlled by AB SCIEX Analyst® software, version 1.6.2 (AB Sciex). Mass spectrometer conditions were ion spray voltage 5,250 volts, turbo gas temperature 700° C., gas 1=65, gas 2=60, curtain gas=20, collision-associated dissociation gas medium and unit mass resolution for Q1 and Q3. Nitrogen was used for all gases and voltages for maximum parent/fragment ion pair intensities were optimized using direct infusion and flow injection analysis. Calibration curves were generated using analyte/IS area response ratios versus nominal concentrations (ng/mL) and weighted linear regressions with a weighting factor of 1/concentration². The IS used for T, ASD and DHEA was d₃-T and d₃-DHT was used for DHT and AND. Back-calculated concentrations were generated using the formula x=(y−b)/m where x is the back-calculated concentration, y is analyte/IS ratio, b is y-intercept and m is slope. Calibrator and quality control acceptance criteria required all acceptable concentrations to have accuracy deviations≤15% from the nominal concentration and relative standard deviation criteria (% RSD)≤15%, except at the lower limit of quantitation (LLOQ), listed in Table 2, which was allowed 20% deviation for both parameters. Values below the LLOQ (BLQ) were treated as 0.

### Results

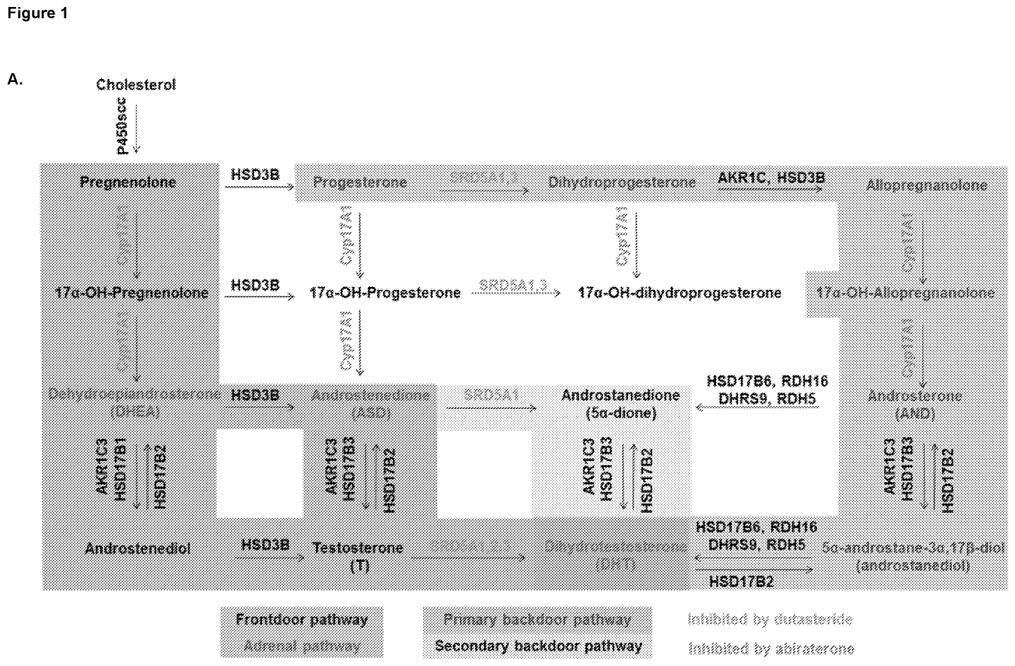

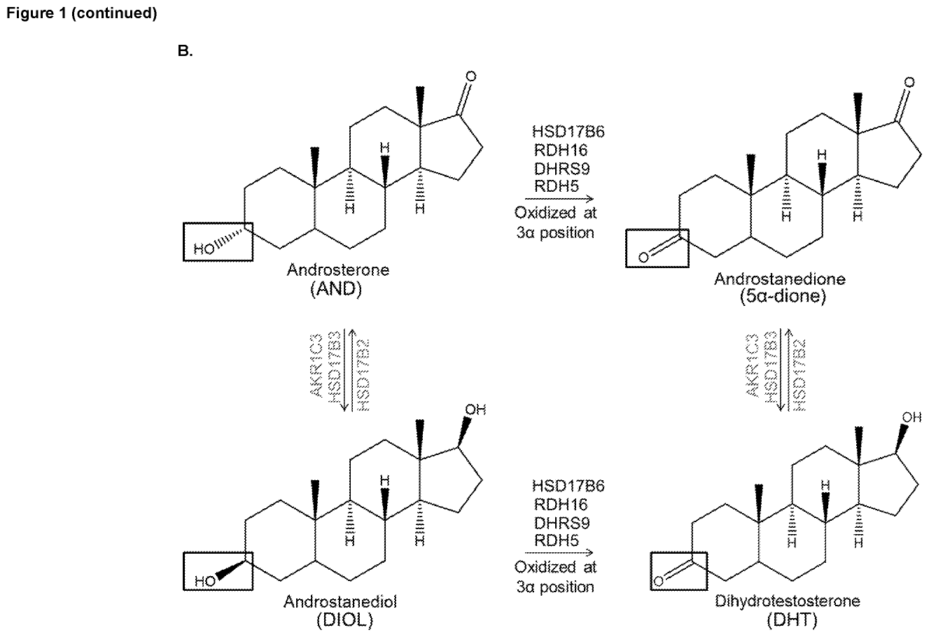

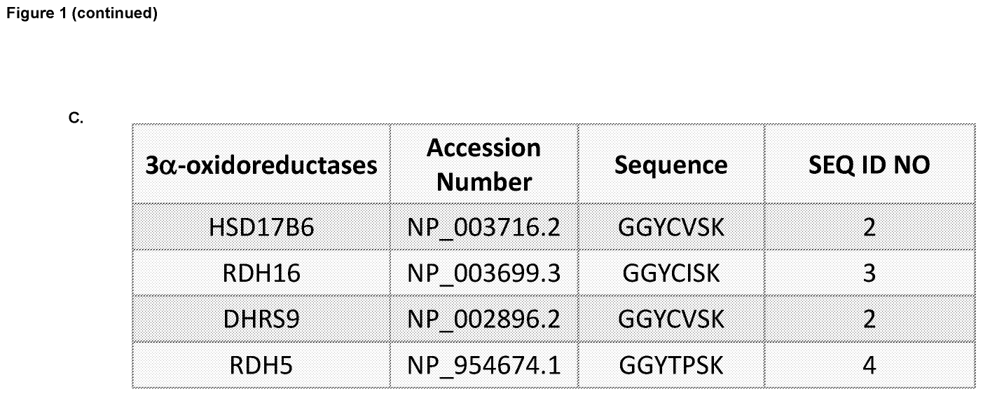

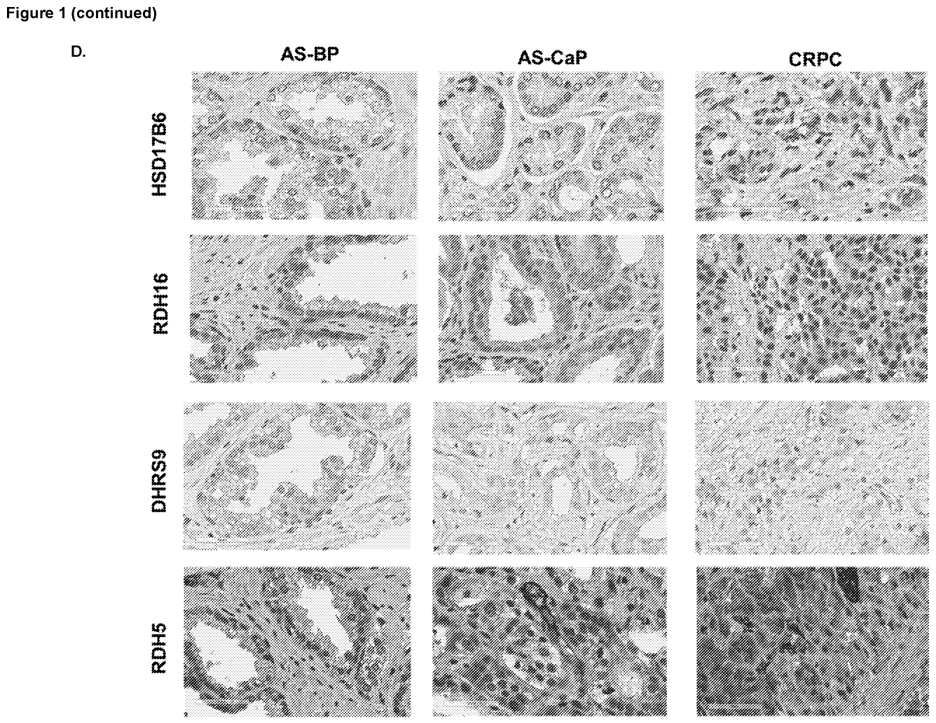

3α-oxidoreductase enzymes share a conserved catalytic site. The primary backdoor pathway uses one or more of four 3α-oxidoreductases to convert AND to 5α-dione or DIOL to DHT (FIG. 1B). DHT synthesis from adrenal androgens is thought to contribute to the development and growth of CRPC. To circumvent issues relating to enzyme redundancy and/or expression of more than 1 enzyme, we used an approach to inhibit all four 3α-oxidoreductases. COBALT protein sequence analysis showed that the four 3α-oxidoreductases shared a common catalytic site (FIG. 1C).

HSD17B6, RDH16, DHRS9 and RDH5 were expressed in clinical CaP. IHC was performed using TMAs that contain clinical specimens of AS-BP, AS-CaP and CRPC collected from 72 patients to assess 3α-oxidoreductase expression. IHC showed that HSD17B6, RDH16, DHRS9 and RDH5 were expressed in AS-BP, AS-CaP and CRPC (FIG. 1D). DHRS9 was expressed only in the cytoplasm. Nuclear expression levels of HSD17B6 and RDH16, but not RDH5, were higher in CRPC tissues than in AS-BP or AS-CaP tissues (FIG. 1E and Table 4). RDH16 levels were higher in the nucleus than the cytoplasm (FIG. 1E and Table 4). Peri-nuclear enhancement was observed for each 3α-oxidoreductase in AS-BP, AS-CaP and CRPC tissues, except for DHRS9.

TABLE 4
Statistical comparisons of 3α-oxidoreductase cytosol and
nuclear protein expression among tissue types; Related to FIG. 1

| | | AS-BP vs | AS-BP vs | AS-CaP vs |
|---|---|---|---|---|
| | | AS-CaP | CRCP | CRCP |
| HSD17B6 | Cytosol | 0.988 | 0.253 | 0.647 |
| | Nuclear | 1.000 | <0.001 | <0.001 |
| RDH16 | Cytosol | 0.384 | 0.096 | 1.000 |
| | Nuclear | 1.000 | <0.001 | <0.001 |
| DHRS9 | Cytosol | 0.502 | 0.501 | 0.095 |
| RDH5 | Cytosol | 1.000 | 0.700 | 0.920 |
| | Nuclear | 0.993 | 0.999 | 1.000 |

Statistical comparisons of 3α-oxidoreductase protein
expression between cystosol and nucleus; Related to FIG. 1

| | | AS-BP | AS-CaP | CRPC |
|---|---|---|---|---|
| HSD17B6 | Cytosol vs. Nuclear | 0.994 | 0.449 | 0.807 |
| RDH16 | Cytosol vs. Nuclear | <0.001 | <0.001 | <0.001 |
| RDH5 | Cytosol vs. Nuclear | 0.023 | 0.890 | 1.000 |

Tukey-Kramer Adjusted P-values

TABLE 5
Comparisons of enzyme expression among cell lines. Related to FIG; 2

| | VCaP | VCaP | VCaP | VCaP | VCaP | VCaP | VCaP | VCaP |
|---|---|---|---|---|---|---|---|---|
| | vs. | vs. | vs. | vs. | vs. | vs. | vs. | vs. |
| Cell line | LNCaP | LAPC-4 | C4-2 | PC-3 | DU145 | CWR-R1 | CWR22 | rCWR22 |
| HSD17B6 | <0.001 | <0.001 | <0.001 | <0.001 | <0.001 | <0.001 | <.001 | <.001 |
| RDH16 | <0.001 | <0.001 | <0.001 | <0.001 | <0.001 | <0.001 | <0.001 | <0.001 |
| DHRS9 | 0.998 | 0.278 | 1 | <0.001 | 1 | 1 | 0.999 | 1 |
| RDH5 | <0.001 | <0.001 | <0.001 | <0.001 | <0.001 | <0.001 | <0.001 | <0.001 |
| SRD5A1 | 0.999 | 0.998 | <0.001 | 0.109 | <0.001 | <0.001 | 0.998 | 0.088 |
| SRD5A2 | 0.999 | 0.901 | 0.999 | 1 | 1 | 1 | 0.005 | 0.401 |
| SRD5A3 | <0.001 | <0.001 | 0.575 | 1 | <0.001 | <0.001 | <0.001 | <0.001 |
| AR | <0.001 | <0.001 | <0.001 | <0.001 | <0.001 | 0.218 | <0.001 | <0.001 |

| | LNCaP | LNCaP | LNCaP | LNCaP | LNCaP | LNCaP | LNCaP |
|---|---|---|---|---|---|---|---|
| | vs. | vs. | vs. | vs. | vs. | vs. | vs. |
| Cell lines | LAPC-4 | C4-2 | PC-3 | DU145 | CWR-R1 | CWR22 | rCWR22 |
| HSD17B6 | <0.001 | 0.002 | <0.001 | <0.001 | 0.005 | 0.553 | 0.395 |
| RDH16 | <0.001 | <0.001 | <0.001 | 0.048 | 0.212 | 0.053 | <0.001 |
| DHRS9 | 0.69 | 1 | <0.001 | 1 | 1 | 1 | 1 |
| RDH5 | 0.001 | 0.667 | <0.001 | <0.001 | <0.001 | <0.001 | <0.001 |
| SRD5A1 | 0.909 | <0.001 | 0.384 | <0.001 | <0.001 | 0.902 | 0.016 |
| SRD5A2 | 0.999 | 1 | 0.999 | 0.987 | 0.999 | 0.091 | 0.904 |
| SRD5A3 | <0.001 | <0.001 | <0.001 | <0.001 | 0.221 | <0.001 | 1 |
| AR | 0.084 | 0.988 | 0.003 | 0.003 | <0.001 | <0.001 | 0.302 |

| | LAPC-4 | LAPC-4 | LAPC-4 | LAPC-4 | LAPC-4 | LAPC-4 |
|---|---|---|---|---|---|---|
| | vs. | vs. | vs. | vs. | vs. | vs. |
| Cell lines | C4-2 | PC-3 | DU145 | CWR-R1 | CWR22 | rCWR22 |
| HSD17B6 | 0.998 | 0.028 | 1 | <0.001 | <0.001 | 0.131 |
| RDH16 | 0.746 | 0.333 | <0.001 | <0.001 | <0.001 | 0.631 |
| DHRS9 | 0.824 | <0.001 | 0.39 | 0.595 | 0.629 | 0.585 |
| RDH5 | 0.207 | <0.001 | <0.001 | <0.001 | <0.001 | <.001 |
| SRD5A1 | <0.001 | 0.016 | <0.001 | <0.001 | 1 | 0.384 |
| SRD5A2 | 0.998 | 0.864 | 0.687 | 0.893 | 0.193 | 0.995 |
| SRD5A3 | <0.001 | <0.001 | <0.001 | <0.001 | <0.001 | <0.001 |
| AR | 0.525 | 0.962 | 0.962 | <0.001 | <0.001 | 1 |

| | C4-2 | C4-2 | C4-2 | C4-2 | C4-2 |
|---|---|---|---|---|---|
| | vs. | vs. | vs. | vs. | vs. |
| Cell lines | PC-3 | DU145 | CWR-R1 | CWR22 | rCWR22 |
| HSD17B6 | 0.003 | 0.993 | <0.001 | <0.001 | 0.513 |
| RDH16 | 0.004 | 0.097 | 0.018 | 0.089 | 1 |
| DHRS9 | <0.001 | 1 | 1 | 1 | 1 |
| RDH5 | <0.001 | <0.001 | <0.001 | <0.001 | <0.001 |
| SRD5A1 | <0.001 | <0.001 | <0.001 | <0.001 | 0.081 |
| SRD5A2 | 0.998 | 0.98 | 0.999 | 0.031 | 0.804 |
| SRD5A3 | 0.317 | <.001 | <0.001 | <0.001 | <0.001 |
| AR | 0.051 | 0.051 | <0.001 | <0.001 | 0.88 |

| | PC-3 | PC-3 | PC-3 | PC-3 |
|---|---|---|---|---|
| | vs. | vs. | vs. | vs. |
| Cell lines | DU145 | CWR-R1 | CWR22 | rCWR22 |
| HSD17B6 | 0.003 | 0.993 | <0.001 | <0.001 |
| RDH16 | <0.001 | <0.001 | <0.001 | 0.002 |
| DHRS9 | <0.001 | <0.001 | <0.001 | <0.001 |
| RDH5 | 0.021 | <0.001 | <0.001 | <0.001 |
| SRD5A1 | <0.001 | <0.001 | 0.015 | <0.001 |
| SRD5A2 | 1 | 1 | 0.003 | 0.347 |
| SRD5A3 | <0.001 | <.001 | <0.001 | <0.001 |
| AR | 1 | <.001 | <0.001 | 0.708 |

| | DU145 | DU145 | DU145 |
|---|---|---|---|
| | vs. | vs. | vs. |
| Cell lines | CWR-R1 | CWR22 | rCWR22 |
| HSD17B6 | <0.001 | <0.001 | 0.098 |
| RDH16 | 0.999 | 1 | 0.146 |
| DHRS9 | 1 | 1 | 1 |
| RDH5 | <.001 | <0.001 | <0.001 |
| SRD5A1 | 0.999 | <0.001 | <0.001 |
| SRD5A2 | 1 | 0.001 | 0.192 |
| SRD5A3 | <0.001 | <0.001 | <.001 |
| AR | <0.001 | <0.001 | 0.708 |

Tukey-Kramer adjustment P-values

### 3α-Oxidoreductase Gene Expression Varied Among CaP Cell Lines

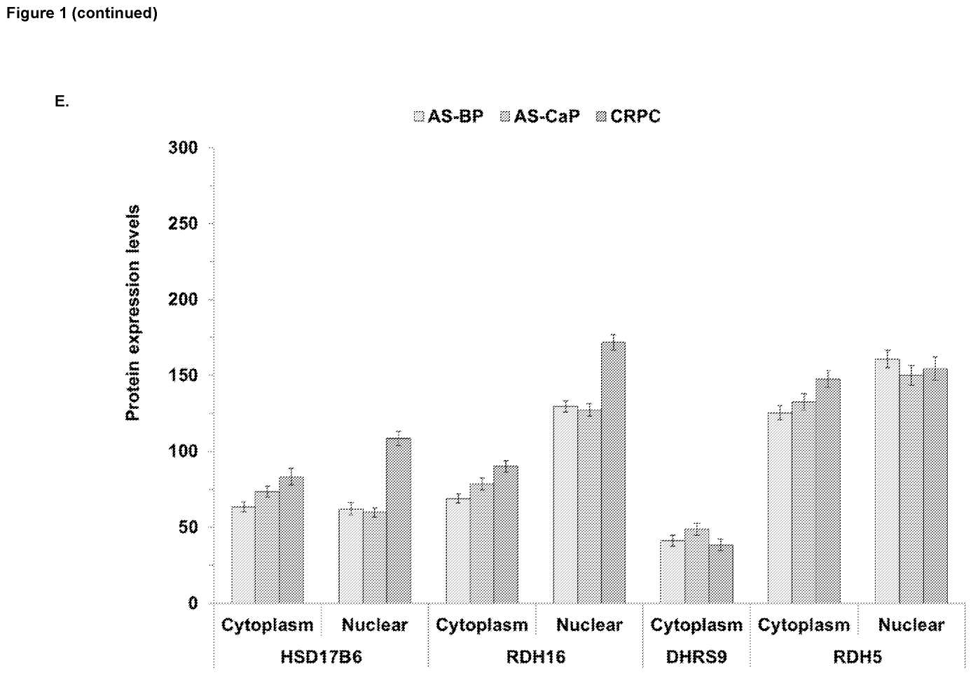

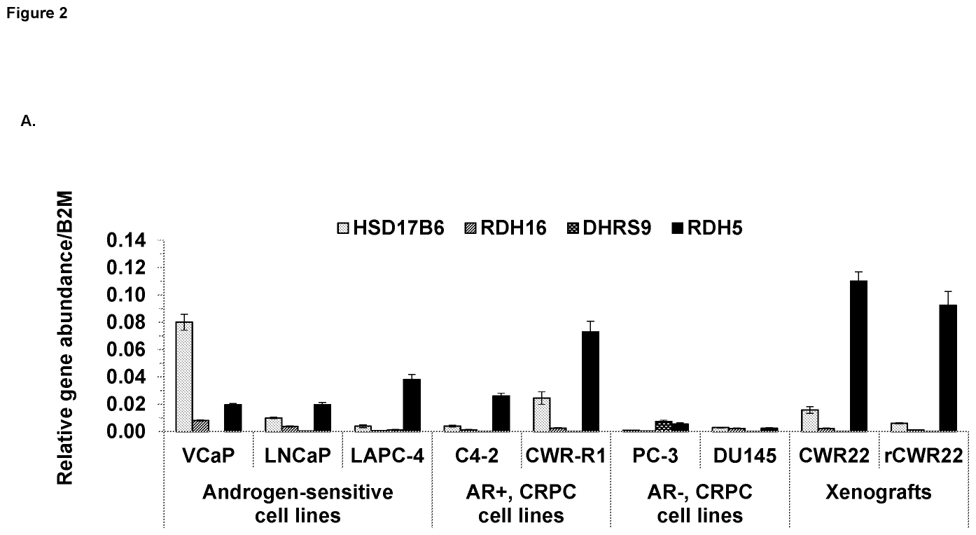

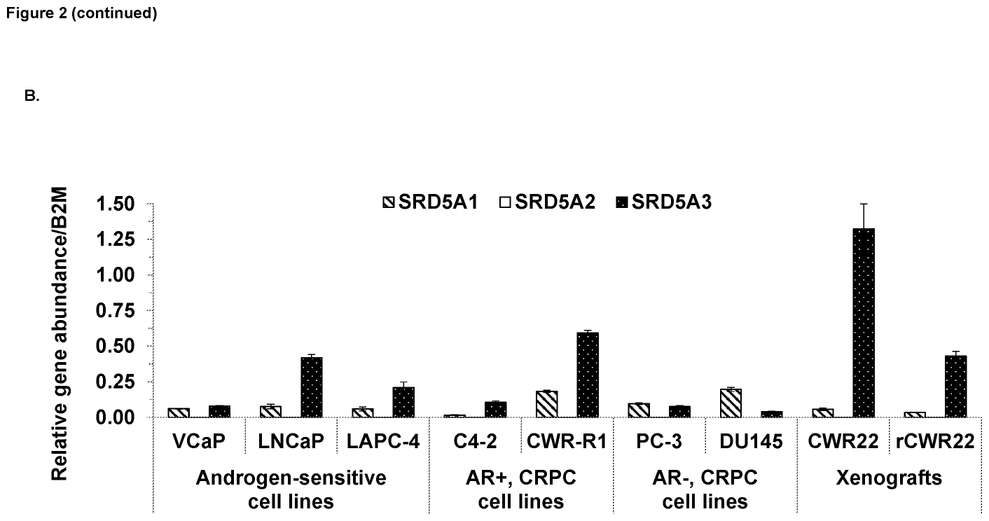

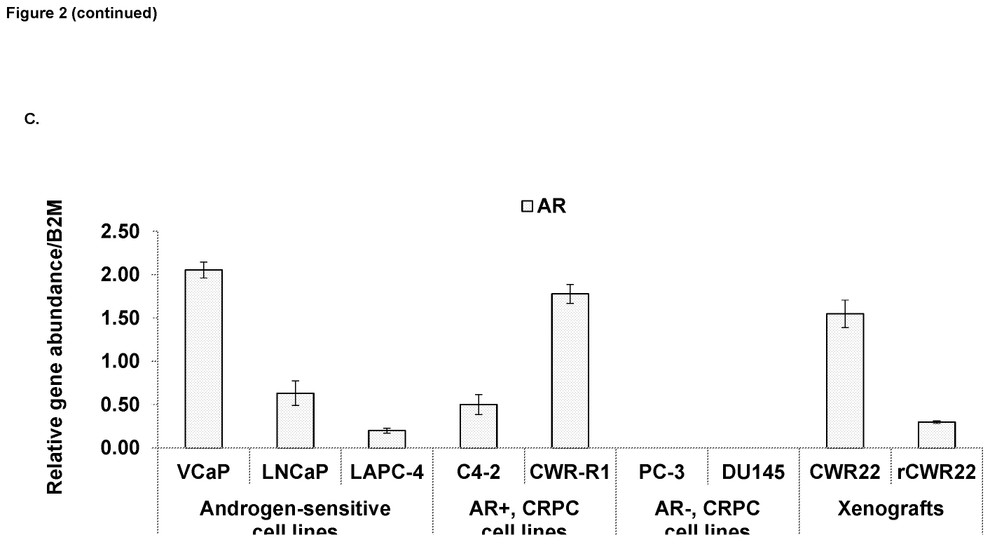

Although 3α-oxidoreductases were detected in clinical samples using IHC, they were not detectable in CaP cell lines using western blot analysis. Therefore, qRT-PCR was performed to determine 3α-oxidoreductase, SRD5A and AR gene expression profiles in CaP cell lines and CWR22 and rCWR22 human CaP xenografts. RDH5 mRNA levels were higher than the expression in the other three 3α-oxidoreductases in all cell lines except VCaP, PC-3 and DU145 cells (FIG. 2A). SRD5A3 mRNA levels were higher than expression in SRD5A1, except in PC-3 and DU145 cells (FIG. 2B). SRD5A2 mRNA was not measurable and clinical specimens. AR mRNA was expressed in all CaP cell lines, except for PC-3 and DU145 cells, and in both xenografts (FIG. 2C). The data indicates that analysis of 3α-oxidoreductase activity in human CaP cell lines required transient expression.

### DHT Levels Increased When RDH16, DHRS9 or RDH5 Were Expressed in LAPC-4 Cells

The effect of 3α-oxidoreductase expression on DHT levels was determined in the androgen-sensitive LAPC-4 cell line that expressed wild-type AR and RDH5 (FIG. 2A) and exhibited 5α-reductase activity. VCaP (S2) and LNCaP cells were not used initially because conversion of T to DHT was not detected. Wild-type 3α-oxidoreductases were expressed transiently in LAPC-4 cells. Results were compared to cells transfected with empty plasmid that were expected to have low endogenous 3α-oxidoreductase activity based on endogenous RDH5 in LAPC-4 cells (FIG. 2A). LAPC-4 cells were treated with DIOL, AND or T. DIOL and AND were used for treatment because 3α-oxidoreductases convert DIOL to DHT or AND to 5α-dione (FIG. 1B). T treatment was used as a control condition because 3α-oxidoreductases do not convert T to DHT.

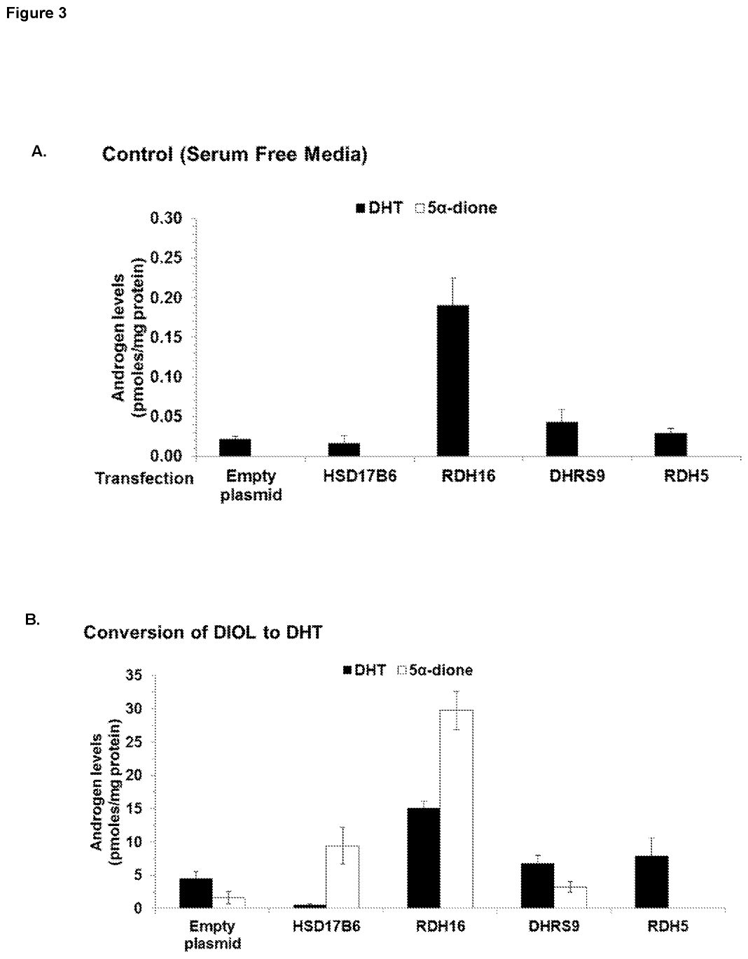

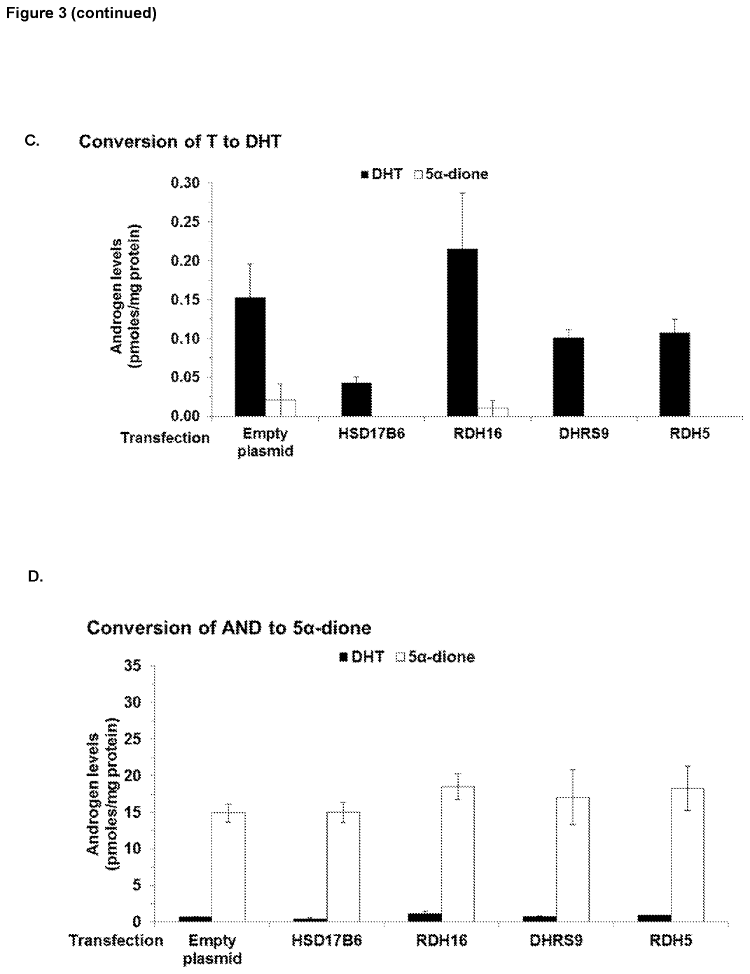

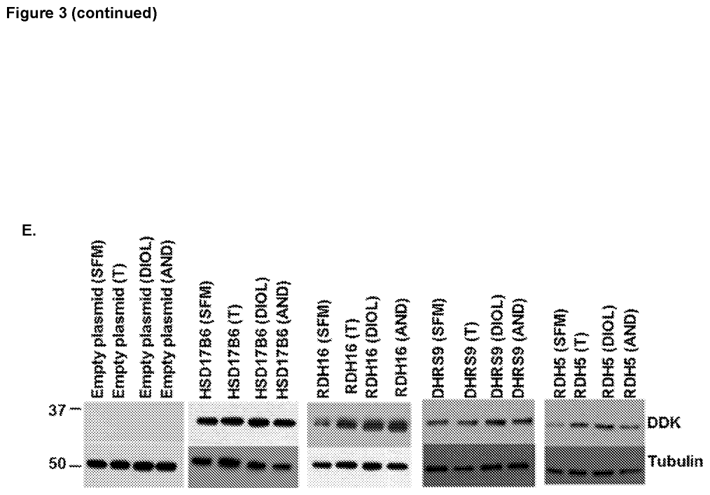

LC-MS/MS analysis revealed that LAPC-4 cells transfected with empty plasmid in SFM (medium without exogenous androgen) produced low levels of DHT (0.0211 pmoles/mg protein) (FIG. 3A empty plasmid and 9A and E). This finding is consistent with qRT-PCR data (FIG. 2A) that LAPC-4 cells express endogenous RDH5 that metabolized DIOL to DHT. DHT levels were higher (p=0.008) in SFM treated LAPC-4 cells that expressed RDH16 compared to SFM treated LAPC-4 cells with empty plasmid (FIG. 3A (RDH16 vs. empty plasmid) and Table 6), which suggested that RDH16 enhanced LAPC-4 cell DHT synthesis in LAPC-4 cells.

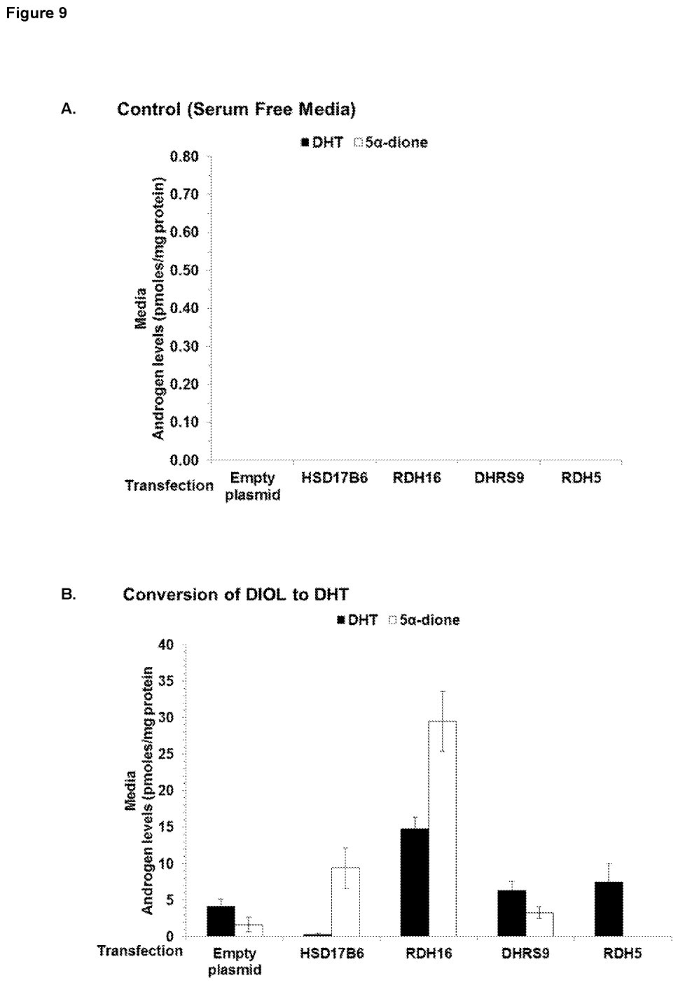

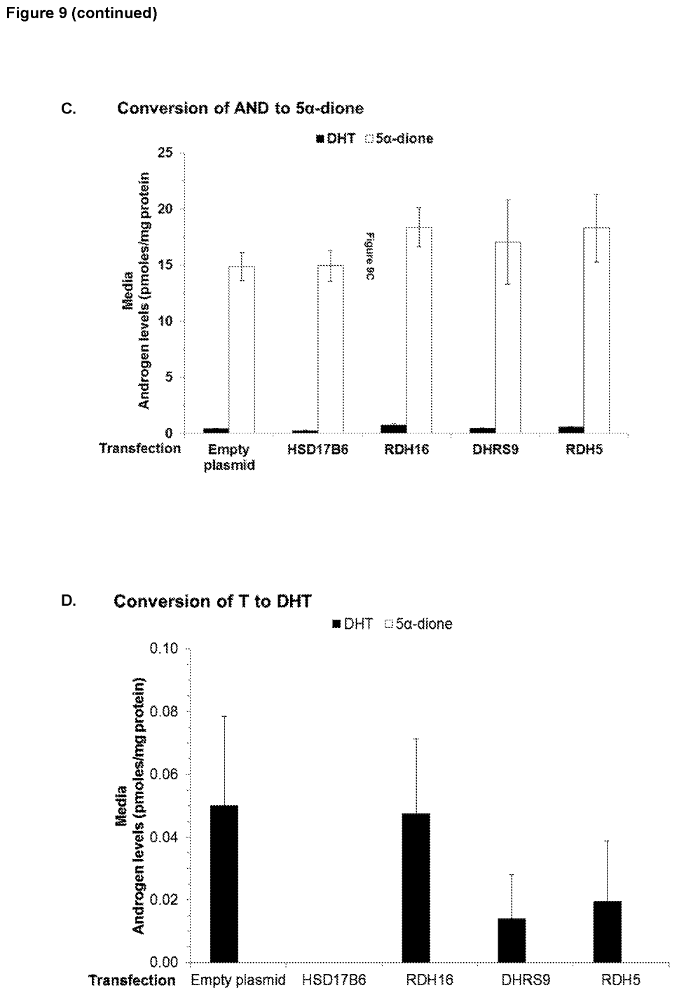

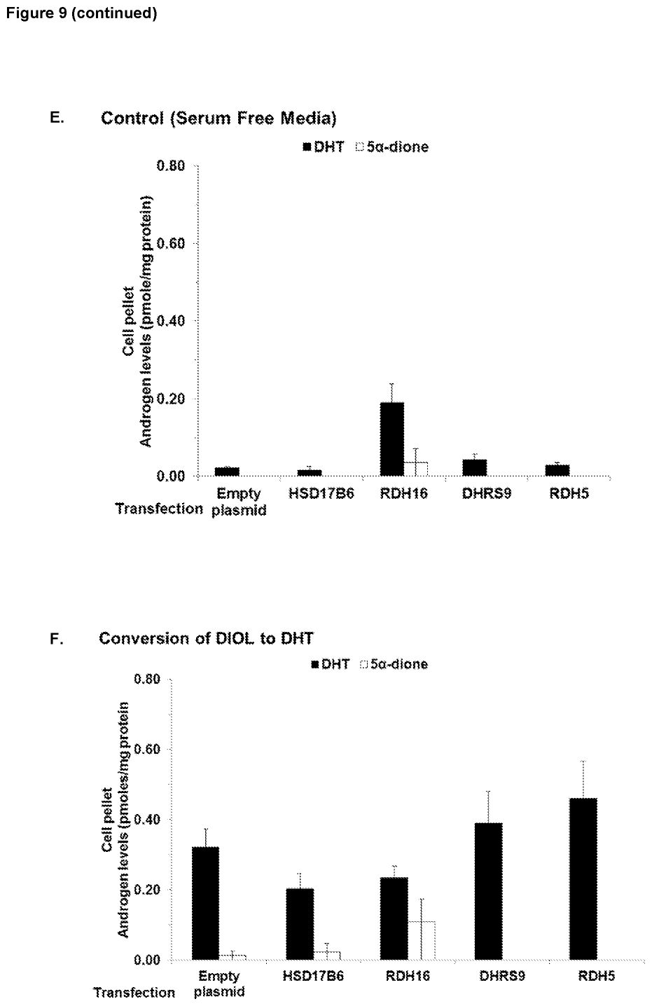

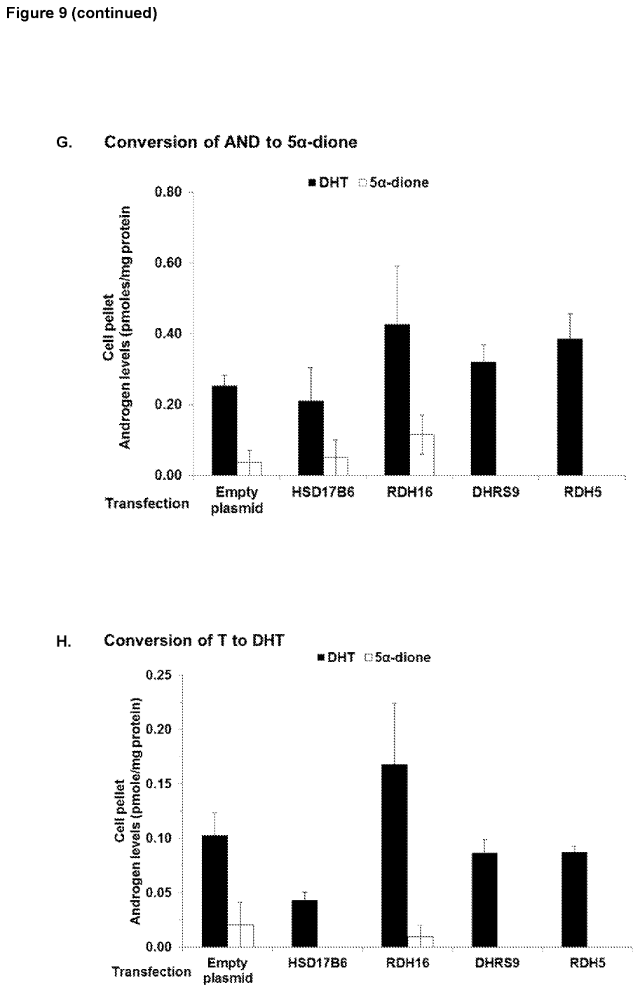

LAPC-4 cells with empty plasmid treated with DIOL produced higher levels of DHT compared to SFM treated LAPC-4 cells with empty plasmid (FIG. 3B; note the change in Y axis between panels A and B; Table 6). DIOL treated LAPC-4 cells that expressed RDH16 produced higher (p=0.022) levels of DHT than SFM treated LAPC-4 cells with empty plasmid (FIG. 3A, FIG. 9B, 9F; Table 6). DIOL treated LAPC-4 cells that expressed HSD17B6 produced lower DHT levels (p<0.001) compared to LAPC-4 cells with empty plasmid. DHT levels appeared to be higher in DIOL treated LAPC-4 that expressed DHRS9 or RDH5 compared to LAPC-4 cells with empty plasmid.

TABLE 6
Comparisons between treatment and SFM; Related to FIG. 3 (Composite)

| | | Treatments compared | | | | | |
|---|---|---|---|---|---|---|---|
| | | DIOL (B) vs. SFM (A) | | T (C) vs. SFM (A) | | AND (D) vs. SFM (A) | |
| | | Analyte | | | | | |
| | | | 5α- | | 5α- | | 5α- |
| | | DHT | dione | DHT | dione | DHT | dione |
| Empty | Empty | <0.001 | 0.072 | 0.024 | 1.000 | <0.001 | <0.001 |
| plasmid or | plasmid | | | | | | |
| enzyme | HSD17B6 | 0.005 | <0.001 | 0.942 | 1.000 | 0.009 | <0.001 |
| | RDH16 | <0.001 | <0.001 | 0.994 | 0.946 | 0.007 | <0.001 |
| | DHRS9 | <0.001 | <0.001 | 0.173 | 1.000 | <0.001 | <0.001 |
| | RDH5 | <0.001 | 1.000 | 0.069 | 1.000 | <0.001 | <0.001 |

Comparisons between 3α-oxidoreductases and empty
plasmid for androgens; Related to FIG. 3 (Composite)

| | | Treatment | | | | | | | |
|---|---|---|---|---|---|---|---|---|---|
| | | SFM (A) | | DIOL (B) | | T (C) | | AND (D) | |
| | | Analyte | | | | | | | |
| | | | 5α- | | 5α- | | 5α- | | 5α- |
| | | DHT | dione | DHT | dione | DHT | dione | DHT | dione |
| Comparisons | HSD17B6 | 0.556 | — | <0.001 | 0.022 | 0.124 | 0.469 | 0.270 | 1.000 |
| | vs. empty | | | | | | | | |
| | RDH16 vs. | 0.008 | — | 0.022 | 0.003 | 0.670 | 0.880 | 0.292 | 0.664 |
| | empty | | | | | | | | |
| | DHRS9 vs. | 0.570 | — | 0.578 | 0.232 | 0.698 | 0.469 | 0.985 | 0.969 |
| | empty | | | | | | | | |
| | RDH5 vs. | 0.928 | — | 0.470 | 0.053 | 0.778 | 0.469 | 0.710 | 0.768 |
| | empty | | | | | | | | |

Dunnett Adjusted P-values

TABLE 7
Comparisons between treatment and SFM (Media); Related to FIG. 3

| | | Treatments compared | | | | | |
|---|---|---|---|---|---|---|---|
| | | DIOL (B) vs. SFM (A) | | T (C) vs. SFM (A) | | AND (D) vs. SFM (A) | |
| | | Analyte | | | | | |
| | | | 5α- | | 5α- | | 5α- |
| | | DHT | dione | DHT | dione | DHT | dione |
| Empty | Empty | <0.001 | 0.081 | 0.278 | 1.000 | <0.001 | 0.001 |
| plasmid or | HSD17B6 | 0.019 | <0.001 | 1.000 | 1.000 | 0.049 | <0.001 |
| Enzyme | RDH16 | <0.001 | <0.001 | 0.472 | 1.000 | <0.001 | <0.001 |
| | DHRS9 | <0.001 | <0.001 | 0.833 | 1.000 | <0.001 | <0.001 |
| | RDH5 | <0.001 | 1.000 | 0.875 | 1.000 | <0.001 | <0.001 |

Comparisons between treatment and SFM (Cell pellet); Related to FIG. 3

| | | | | | | | |
|---|---|---|---|---|---|---|---|
| Empty | Empty | <0.001 | 0.072 | 0.002 | 1.000 | <0.001 | <0.001 |
| plasmid or | HSD17B6 | 0.005 | <0.001 | 0.120 | 1.000 | 0.006 | <0.001 |
| Enzyme | RDH16 | 0.869 | <0.001 | 0.963 | 0.994 | 0.278 | <0.001 |
| | DHRS9 | <0.001 | <0.001 | 0.102 | 1.000 | 0.001 | <0.001 |
| | RDH5 | <0.001 | 1.000 | 0.007 | 1.000 | <0.001 | <0.001 |

Comparisons between 3α-oxidoreductases and empty
plasmid for androgens (Media); Related to FIG. 3

| | | Treatment | | | | | | | |
|---|---|---|---|---|---|---|---|---|---|
| | | SFM (A) | | DIOL (B) | | T (C) | | AND (D) | |
| | | Analyte | | | | | | | |
| | | | 5α- | | 5α- | | 5α- | | 5α- |
| | | DHT | dione | DHT | dione | DHT | dione | DHT | dione |
| Comparisons | HSD17B6 | — | — | <0.001 | 0.024 | — | — | 0.072 | 1.000 |
| | vs. empty | | | | | | | | |
| | RDH16 vs. | — | — | 0.366 | 0.004 | — | — | 0.086 | 0.671 |
| | empty | | | | | | | | |
| | DHRS9 vs. | — | — | 0.838 | 0.234 | — | — | 1.000 | 0.973 |
| | empty | | | | | | | | |
| | RDH5 vs. | — | — | 0.884 | 0.068 | — | — | 0.654 | 0.769 |
| | empty | | | | | | | | |

Comparisons between 3α-oxidoreductases and empty
plasmid for androgens (Cell pellet); Related to FIG. 3

| | | Treatment | | | | | | | |
|---|---|---|---|---|---|---|---|---|---|
| | | SFM (E) | | DIOL (F) | | T (G) | | AND (H) | |
| | | Analyte | | | | | | | |
| | | | 5α- | | 5α- | | 5α- | | 5α- |
| | | DHT | dione | DHT | dione | DHT | dione | DHT | dione |
| Comparisons | HSD17B6 | 0.556 | — | 0.344 | 0.013 | 0.047 | 0.469 | 0.765 | 1.000 |
| | vs. empty | | | | | | | | |
| | RDH16 vs. | 0.008 | — | 0.678 | <0.001 | 0.409 | 0.880 | 0.718 | 0.664 |
| | empty | | | | | | | | |
| | DHRS9 vs. | 0.570 | — | 0.932 | 0.253 | 0.956 | 0.469 | 0.931 | 0.969 |
| | empty | | | | | | | | |
| | RDH5 vs. | 0.928 | — | 0.624 | 0.078 | 0.976 | 0.469 | 0.800 | 0.768 |
| | empty | | | | | | | | |

Tukey-Kramer Adjusted P-values

DHT levels were higher (p=0.024) when LAPC-4 cells with empty plasmid were treated with T (FIG. 3C; FIG. 9C; S3G; Tables 6 and 7) compared to SFM treated LAPC-4 cells with empty plasmid, which is consistent with T to DHT conversion by endogenous SRD5A. DHT levels were not significantly different among T treated LAPC-4 cells that expressed 3α-oxidoreductases or LAPC-4 cells with empty plasmid (Table 6).

5α-dione was produced when LAPC-4 cells with empty plasmid were treated with DIOL or AND (FIG. 3B and 3D; FIG. 9B; 9D; 9F; 9H). The data were consistent with the high levels of endogenous RDH5 mRNA found in LAPC-4 cells (FIG. 2A). DIOL treated LAPC-4 cells that transiently expressed HSD17B6, RDH16 or DHRS9 produced 5α-dione, but only LAPC-4 cells that expressed HSD17B6 (p=0.022) or RDH16 (p=0.003) produced higher 5α-dione levels compared to LAPC-4 cells with empty plasmid (FIG. 3B; Table 6). The data suggested LAPC-4 cells endogenously converted DIOL to DHT and HSD17B6 or RDH16 converted AND to 5α-dione. AND treated LAPC-4 cells that transiently expressed 3α-oxidoreductases produced similar 5α-dione levels as AND treated LAPC-4 cells with empty plasmid (FIG. 3D). The data suggested that LAPC-4 cells may not be an appropriate CaP cell model to study the ability for 3α-oxdioredcutases to convert AND to 5α-dione.

Catalytic amino acid substitution or deletion impaired 3α-oxidoreductase activity. Site-directed mutagenesis of the 3α-oxidoreductase catalytic residues confirmed Y176/175 and K179/180 were essential for 3α-oxidoreductase activity for all four enzymes.

The mutants included Δcat (deletion of the catalytic residues) or Y176F for HSD17B6, RDH16, DHRS9 or Y175F for RDH5 and K180R for HSD17B6, RDH16 and DHRS9 or K179R for RDH5. Rationale for the Y→F mutation was rested on their similarity in size but difference in an essential hydroxyl group. This mutation was not expected to alter protein folding but would affect enzyme activity. The K→R mutation was chosen because R would maintain a positive charge but was not expected alter protein folding.

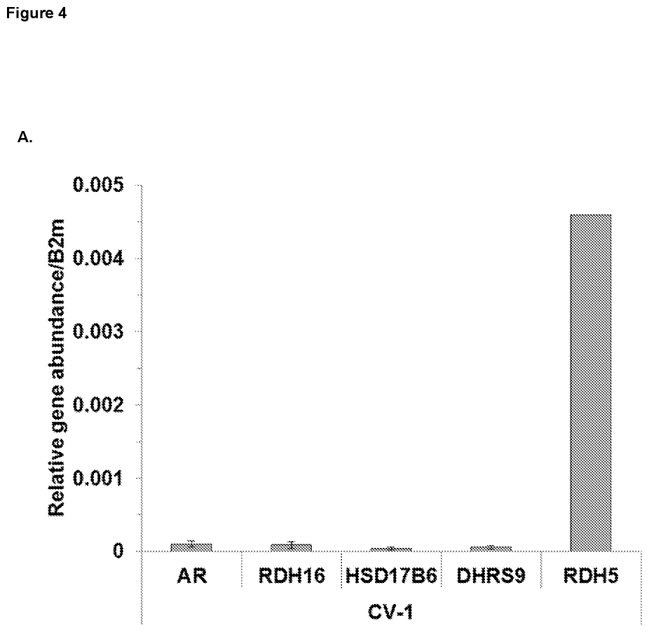

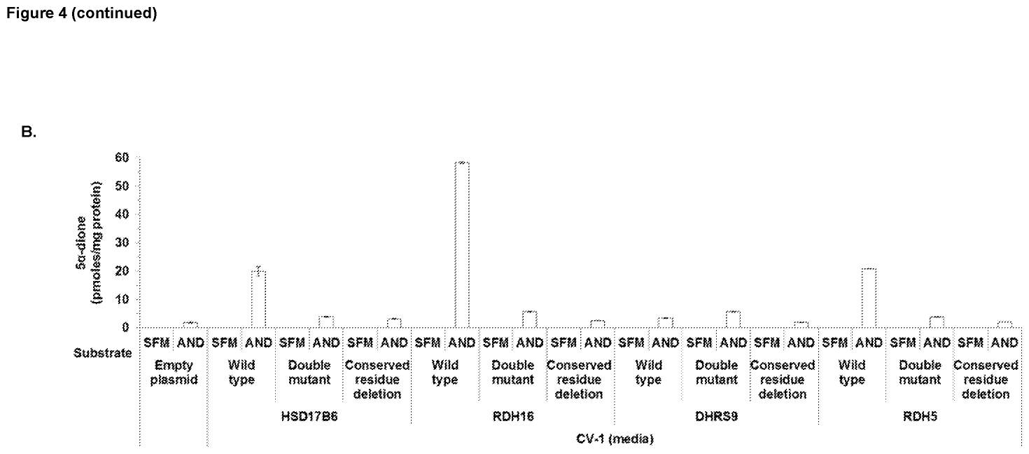

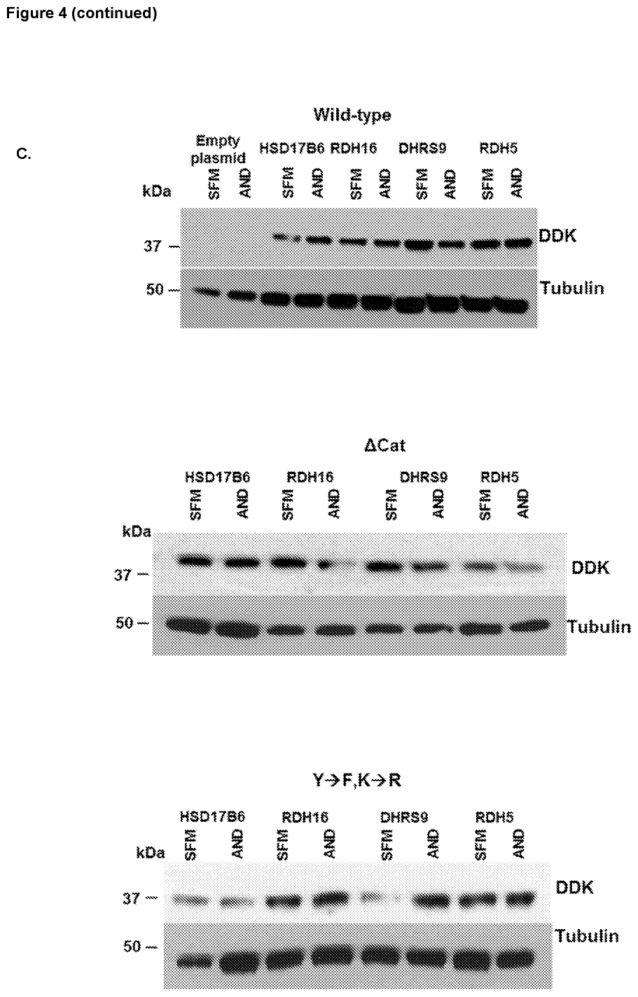

qRT-PCR confirmed that CV-1 cells had low endogenous AR and 3α-oxidoreductase mRNA levels that suggests RDH5 converts AND to 5α-dione in control CV-1 cells (FIG. 4A). 3α-oxidoreductase protein expression was not detected using western blotting (data not shown). Therefore, despite low expression of RDH5, CV-1 cells were used to express wild-type or mutant 3α-oxidoreductases to evaluate the effect of the mutations on the activity of 3α-oxidoreductases.

CV-1 treated with AND produced only a small amount of 5α-dione that was measurable only in the media (FIG. 4B). T or DHT were not detected in the media or CV-1 cell pellets. Therefore, subsequent experiments measured only androgens in media. CV-1 cells that expressed wild-type HSD17B6, RDH16 or RDH5 and were treated with AND produced 5α-dione at levels higher than levels observed in media from CV-1 cells with empty plasmid (Table 8). Δcat or the Y→F, K→R mutations reduced 5α-dione levels to background. Wild-type DHRS9 activity was impaired in CV-1 cells, which suggested CV-1 cells possessed inhibitory mechanisms that interfered with DHRS9 activity (FIG. 4B). 5α-dione levels did not increase in CV-1 cells that expressed Y→F, K→R or Δcat 3α-oxidoreductase mutants. 3α-oxidoreductase enzyme expression was verified using western blot (FIG. 4C). The findings suggested that Y176 (Y175) and K180 (K179) were critical residues for enzyme activity for all three of the 4 3α-oxidoreductases.

TABLE 8
Comparisons between AND and SFM treatments; Related to FIG. 4

| Plasmid type | Enzyme | 5α-dione |
|---|---|---|
| Empty | — | 0.376 |
| Wild-type | HSD17B6 | 0.195 |
| | RDH16 | 0.001 |
| | DHRS9 | 0.039 |
| | RDH5 | <0.001 |
| Δcat | HSD17B6 | 0.039 |
| | RDH16 | 0.042 |
| | DHRS9 | 0.005 |
| | RDH5 | 0.003 |
| Y→F, K→R | HSD17B6 | 0.025 |
| | RDH16 | 0.041 |
| | DHRS9 | 0.005 |
| | RDH5 | 0.001 |

Comparisons between wild-type, Δcat or Y→F, K→R; Related to FIG. 4

| Enzyme | Comparison | 5α-dione |
|---|---|---|
| HSD17B6 | Wild-type vs. Δcat | 0.015 |
| | Wild-type vs. Y→F, K→R | 0.026 |
| RDH16 | Wild-type vs. Δcat | 0.001 |
| | Wild-type vs. Y→F, K→R | <0.001 |
| DHRS9 | Wild-type vs. Δcat | 0.418 |
| | Wild-type vs. Y→F, K→R | 0.021 |
| RDH5 | Wild-type vs. Δcat | <0.001 |
| | Wild-type vs. Y→F, K→R | <0.001 |

Comparisons between Enzyme and empty plasmid; Related to FIG. 4

| Plasmid type | Comparison | 5α-dione |
|---|---|---|
| Wild-type | HSD17B6 vs. empty | <0.001 |
| | RDH16 vs. empty | <0.001 |
| | DHRS9 vs. empty | 0.158 |
| | RDH5 vs. empty | <0.001 |
| Δcat | HSD17B6 vs. empty | 0.048 |
| | RDH16 vs. empty | 0.002 |
| | DHRS9 vs. empty | 0.038 |
| | RDH5 vs. empty | 0.051 |
| Y→F, K→R | HSD17B6 vs. empty | 0.327 |
| | RDH16 vs. empty | 0.810 |
| | DHRS9 vs. empty | 1.000 |
| | RDH5 vs. empty | 0.993 |

Catalytic site deletion (Δcat), double mutation (Y→F, K→R)
Tukey-Kramer Adjusted P-values

TABLE 9

| | | | | | | | |
|---|---|---|---|---|---|---|---|
| Tissue | Compartment | Treatments compared | HSD17B6 | RDH16 | RDH5 | DHRS9 | AR |

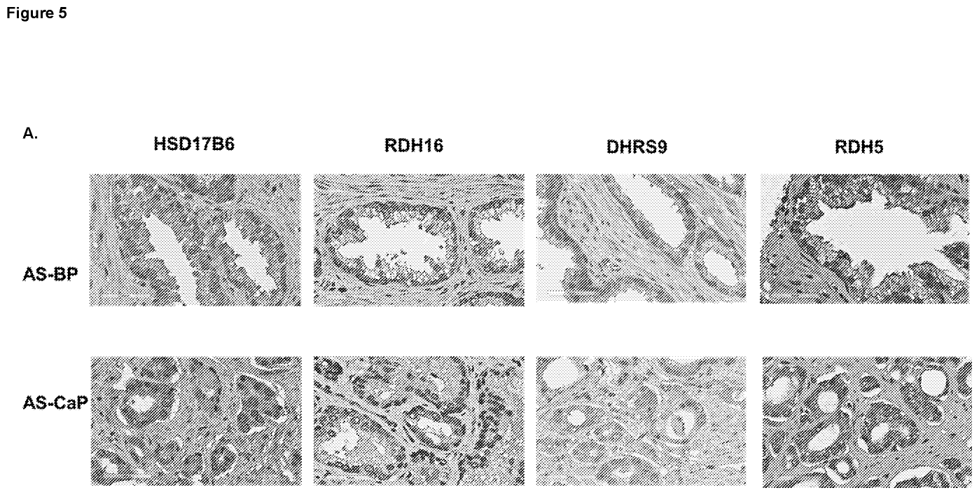

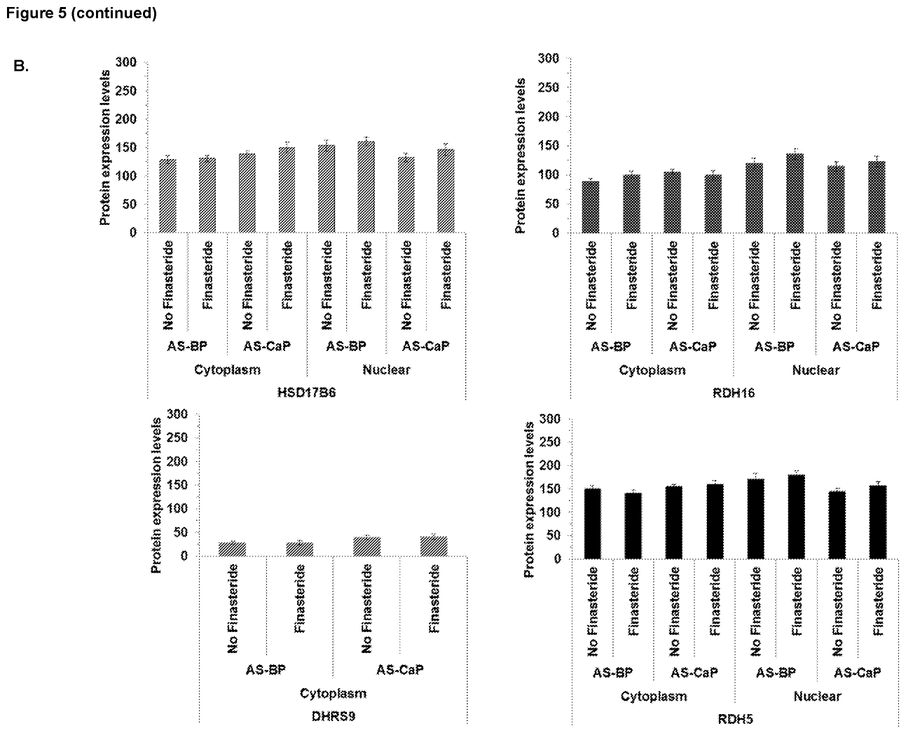

Protein expression level comparison between non-finasteride and finasteride groups; Related to FIG. 5

| | | | | | | | |
|---|---|---|---|---|---|---|---|
| AS-BP | Cytoplasm | No Finasteride - | 1 | 0.989 | 1.000 | 1.000 | — |
| | | Finasteride | | | | | |
| | Nuclear | No Finasteride - | 1 | 0.996 | 1.000 | — | 0.836 |
| | | Finasteride | | | | | |
| AS-CaP | Cytoplasm | No Finasteride - | 0.999 | 1.000 | 1.000 | 0.983 | — |
| | | Finasteride | | | | | |
| | Nuclear | No Finasteride - | 0.988 | 1.000 | 0.994 | — | 0.878 |
| | | Finasteride | | | | | |

Protein expression level comparison by finasteride status and tissue type; Related to FIG. 5

| | | | | | | | |
|---|---|---|---|---|---|---|---|
| Cytoplasm | No Finasteride | AS-BP - CaP | 1 | 0.882 | 1.000 | 0.241 | — |
| | Finasteride | AS-BP - CaP | 0.499 | 1.000 | 0.936 | 0.532 | — |
| Nuclear | No Finasteride | AS-BP - CaP | 0.155 | 1.000 | 0.669 | — | 0.999 |
| | Finasteride | AS-BP - CaP | 0.983 | 1.000 | 0.779 | — | 0.113 |

Tukey-Kramer Adjusted P-values

3α-oxidoreductase expression in AS-BP and CaP specimens from research subjects treated with finasteride. To address the clinical relevance of the 3α-oxidoreductases, LC-MS/MS was applied to tissues obtained from a randomized double-blind placebo-controlled clinical trial of selenium supplementation and finasteride treatment of research subjects with CaP prior to radical prostatectomy (Selenium and Finasteride Pre-Treatment Trial, 1104607). Research subjects who received finasteride alone or in combination with selenium were compared to research subjects who received selenium alone or placebo. Research subjects treated with finasteride had decreased CaP tissue levels of DHT in both benign and malignant macro-dissected samples, although DHT levels remained sufficient to activate AR (data not shown). TMAs generated from the clinical trial were sectioned and analyzed using IHC. HSD17B6, RDH16, DHRS9 and RDH5 were expressed in AS-BP and AS-CaP (FIG. 5A). HSD17B6, RDH16, DHRS9 and RDH5 expression levels and subcellular localization were similar to the 72 research subjects' specimens analyzed previously (FIGS. 1D and 1E). The data suggested that DHT synthesis persisted in spite of finasteride inhibition of SRD5A either from incomplete inhibition of SRD5A or from backdoor DHT synthesis.

Combination dutasteride and 3α-oxidoreductase mutants decreased DHT greater than dutasteride alone. Primary backdoor DHT synthesis may facilitate CaP resistance to 5α-reductase inhibition. Therefore, simultaneous inhibition of the terminal steps of the frontdoor and primary backdoor pathways could lower DHT levels more effectively than targeting either terminal step alone. LAPC-4 cells had SRD5A activity and dutasteride treatment decreased LAPC-4 DHT levels. LAPC-4 cells also were capable of backdoor DHT synthesis using 3α-oxidoreductases to convert DIOL to DHT (FIG. 3A). Therefore, LAPC-4 cells were used to test the effect of inhibition of the terminal steps of frontdoor and primary backdoor androgen pathway. Dutasteride was used to inhibit SRD5A activity and block the frontdoor pathway. Activity impairing mutants were used to block enzymatic activity in the primary backdoor pathway.

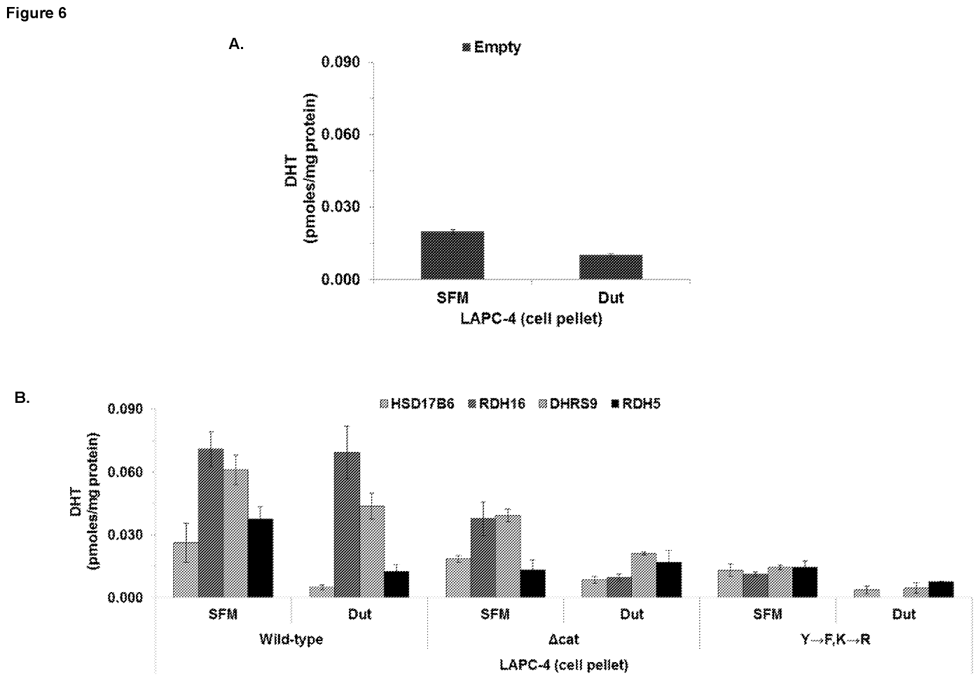

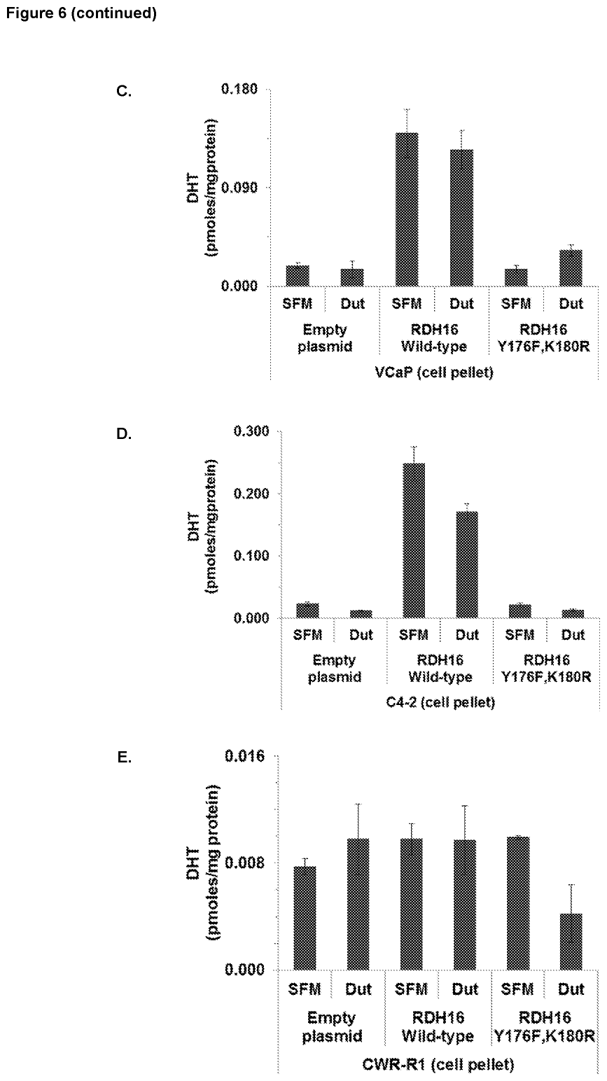

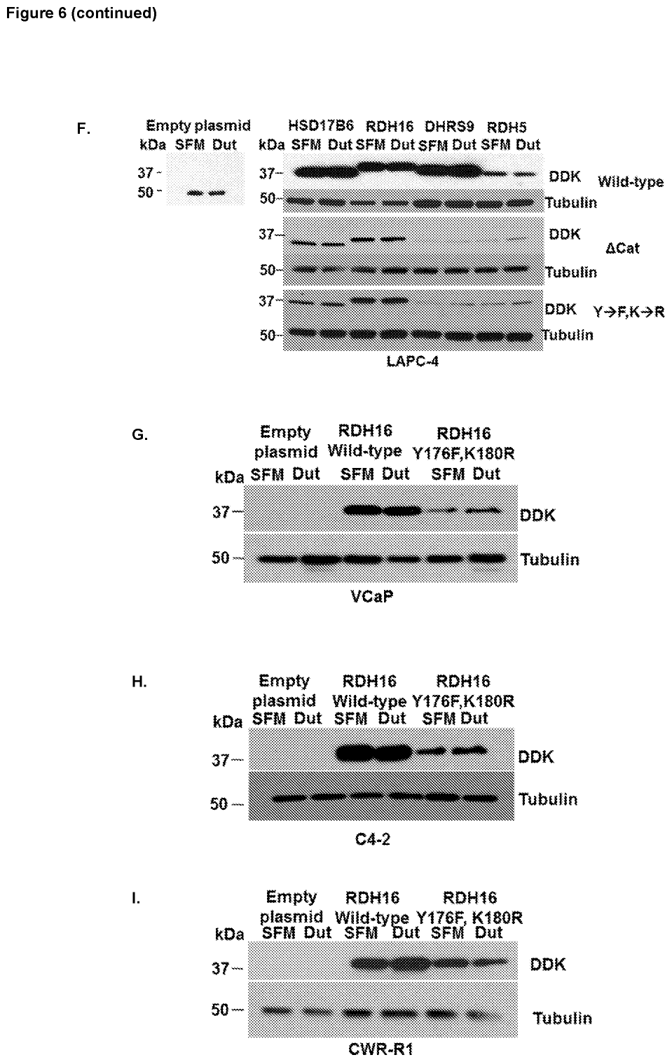

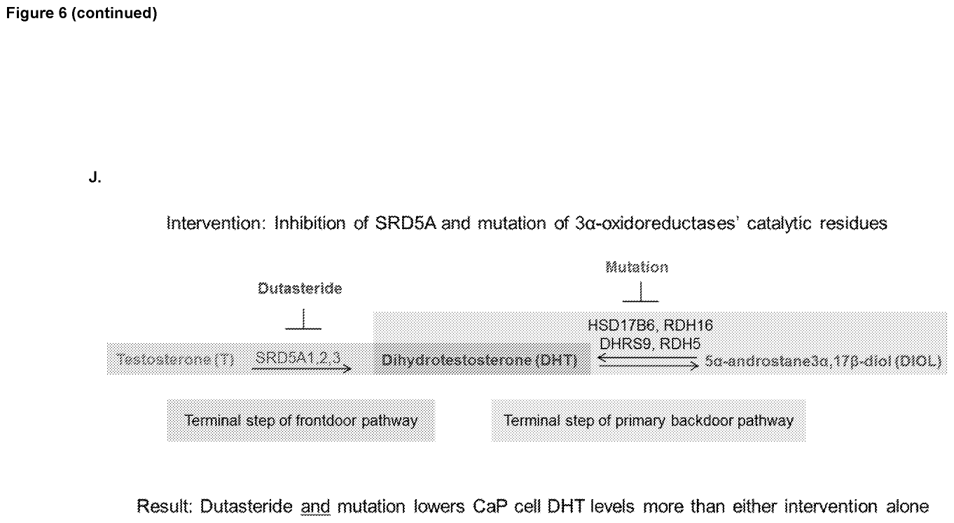

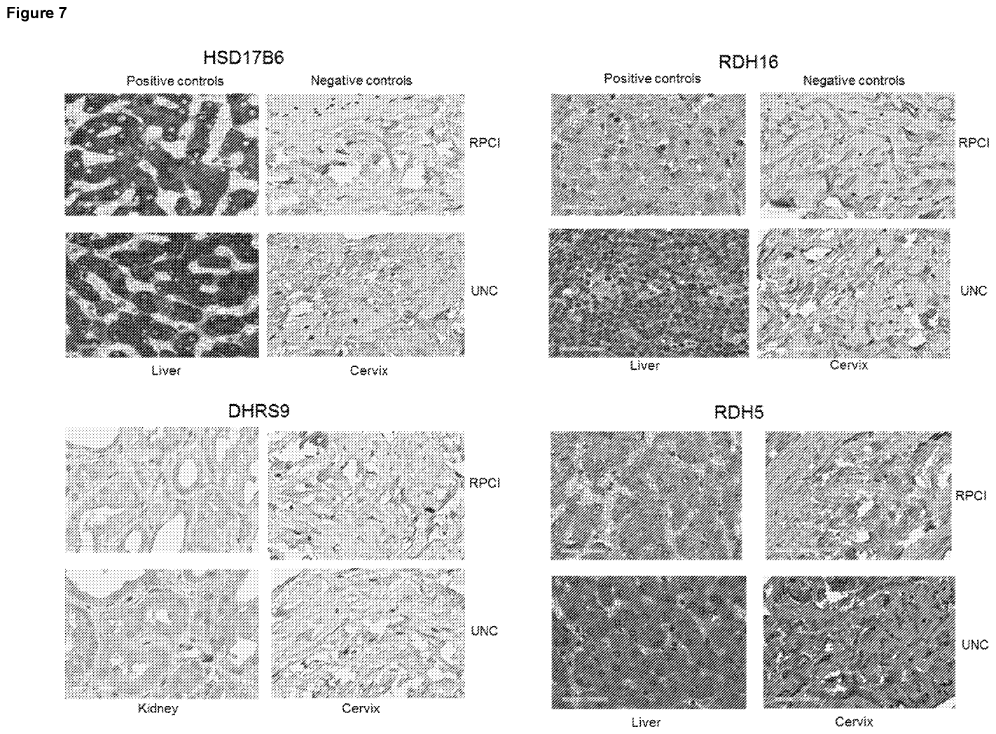

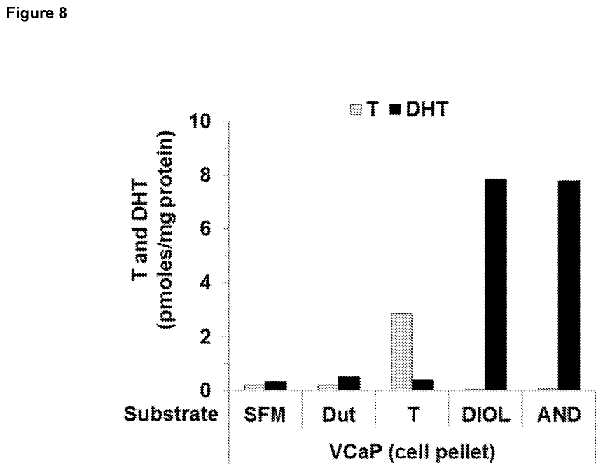

DHT levels were higher in LAPC-4 cell pellets that overexpressed RDH16 (p<0.001) or DHRS9 (p<0.001) compared to LAPC-4 cell pellets with empty plasmid (FIG. 6A [SFM alone]; Table 10); no androgens were measurable in media. DHT levels were lower in dutasteride treated LAPC-4 cells with empty plasmid compared to SFM treated LAPC-4 cells with empty plasmid (FIG. 6A; p=0.041; Table 10). No effect of dutasteride was observed in LAPC-4 cells that expressed wild-type RDH16 or DHRS9, which suggested that RDH16 or DHRS9 was sufficient for primary backdoor DHT synthesis. LAPC-4 cells that expressed Δcat of RDH5 or Y→F, K→R mutants of RDH16 or DHRS9 had lower DHT levels (RDH5 p=0.046; RDH16 p=0.006; DHRS9; p=0.004) compared to LAPC-4 cells that expressed wild-type RDH16, DHRS9 or RDH5. DHT levels were lowered further by dutasteride treatment of LAPC-4 cells that expressed mutant RDH16 or DHRS9 compared to LAPC-4 cells treated with dutasteride alone. DHT levels were significantly lower in LAPC-4 cells that expressed RDH16-Δcat (p=0.008), RDH16-Y176F, K180R (p=0.004), DHRS9-Δcat (p=0.029) or DHRS9-Y176F, K180R (p=0.004) after dutasteride treatment compared to LAPC-4 cells that overexpressed wild-type RDH16 or DHRS9 (FIG. 6A; Table 10). Subsequent experiments focused on RDH16 because [1] wild-type RDH16 expression rendered dutasteride ineffective in LAPC-4 cells and [2] DHT levels were lowered significantly by expression of RDH16-Y176F, K180R and dutasteride treatment.

TABLE 10
Comparisons between dutasteride and SFM treated CaP cell lines; Related to FIG. 6

| Cell line | Plasmid type | Enzyme | DHT |
|---|---|---|---|
| LAPC-4 (A) | Empty | — | 0.041 |
| | Wild-Type | HSD17B6 | 0.242 |
| | | RDH16 | 1.000 |
| | | DHRS9 | 0.315 |
| | | RDH5 | 0.094 |
| | Δcat | HSD17B6 | 0.025 |
| | | RDH16 | 0.138 |
| | | DHRS9 | 0.045 |
| | | RDH5 | 1 |
| | Y→F, K→R | HSD17B6 | 0.051 |
| | | RDH16 | 0.015 |
| | | DHRS9 | 0.159 |
| | | RDH5 | 0.157 |
| VCaP (B) | Empty | — | 0.165 |
| | Wild-Type | RDH16 | 0.029 |
| | Y176F, K180R | RDH16 | 1.000 |
| C4-2 (C) | Empty | — | 0.017 |
| | Wild-Type | RDH16 | 0.151 |
| | Y176F, K180R | RDH16 | 0.108 |
| CWR-R1 (D) | Empty | — | 0.814 |
| | Wild-Type | RDH16 | 0.988 |
| | Y176F, K180R | RDH16 | 0.126 |

Comparisons among wild-type, Δcat and Y→F, K→R mutants; Related to FIG. 6B

| | Enzyme | Treatment | Enzyme Comparison | DHT |
|---|---|---|---|---|
| LAPC-4 (A) | HSD17B6 | SFM | Wild-type vs. Δcat | 0.648 |
| | | | Wild-type vs. Y→F, K→R | 0.326 |
| | RDH16 | SFM | Wild-type vs. Δcat | 0.052 |
| | | | Wild-type vs. Y→F, K→R | 0.006 |
| | DHRS9 | SFM | Wild-type vs. Δcat | 0.057 |
| | | | Wild-type vs. Y→F, K→R | 0.004 |
| | RDH5 | SFM | Wild-type vs. Δcat | 0.046 |
| | | | Wild-type vs. Y→F, K→R | 0.055 |
| | HSD17B6 | Dut | Wild-type vs. Δcat | 0.362 |
| | | | Wild-type vs. Y→F, K→R | 0.846 |
| | RDH16 | Dut | Wild-type vs. Δcat | 0.008 |
| | | | Wild-type vs. Y→F, K→R | 0.004 |
| | DHRS9 | Dut | Wild-type vs. Δcat | 0.029 |
| | | | Wild-type vs. Y→F, K→R | 0.004 |
| | RDH5 | Dut | Wild-type vs. Δcat | 0.735 |
| | | | Wild-type vs. Y→F, K→R | 0.649 |
| VCaP (B) | RDH16 | SFM | Wild-type vs. Y→F, K→R | 0.005 |
| | RDH16 | Dut | Wild-type vs. Y→F, K→R | 0.910 |
| C4-2 (C) | RDH16 | SFM | Wild-type vs. Y→F, K→R | 0.001 |
| | RDH16 | Dut | Wild-type vs. Y→F, K→R | <0.001 |
| CWR-R1 (D) | RDH16 | SFM | Wild-type vs. Y→F, K→R | 0.981 |
| | RDH16 | Dut | Wild-type vs. Y→F, K→R | 0.355 |

Comparisons between 3α-oxidoreductases and empty plasmid

| | Enzyme | Treatment | Comparison | DHT |
|---|---|---|---|---|
| LAPC-4 (A) | HSD17B6 | SFM | Empty vs. Wild-type | 1.000 |
| | | Dut | Empty vs. Wild-type | 1.000 |
| | | SFM | Empty vs. Δcat | 1.000 |
| | | Dut | Empty vs. Δcat | 1.000 |
| | | SFM | Empty vs. Y→F, K→R | 1.000 |
| | | Dut | Empty vs. Y→F, K→R | 1.000 |
| | RDH16 | SFM | Empty vs. Wild-type | <0.001 |
| | | Dut | Empty vs. Wild-type | <0.001 |
| | | SFM | Empty vs. Δcat | 0.550 |
| | | Dut | Empty vs. Δcat | 1.000 |
| | | SFM | Empty vs. Y→F, K→R | 0.999 |
| | | Dut | Empty vs. Y→F, K→R | 0.992 |
| | DHRS9 | SFM | Empty vs. Wild-type | <0.001 |
| | | Dut | Empty vs. Wild-type | 0.002 |
| | | SFM | Empty vs. Δcat | 0.398 |
| | | Dut | Empty vs. Δcat | 0.985 |
| | | SFM | Empty vs. Y→F, K→R | 1.000 |
| | | Dut | Empty vs. Y→F, K→R | 1.000 |
| | RDH5 | SFM | Empty vs. Wild-type | 0.552 |
| | | Dut | Empty vs. Wild-type | 1.000 |
| | | SFM | Empty vs. Δcat | 1.000 |
| | | Dut | Empty vs. Δcat | 1.000 |
| | | SFM | Empty vs. Y→F, K→R | 1.000 |
| | | Dut | Empty vs. Y→F, K→R | 1.000 |
| VCaP (B) | RDH16 | SFM | Empty vs. Wild-type | 0.031 |
| | | Dut | Empty vs. Wild-type | 0.116 |
| | | SFM | Empty vs. Y→F, K→R | 0.229 |
| | | Dut | Empty vs. Y→F, K→R | 0.149 |
| C4-2 (C) | RDH16 | SFM | Empty vs. Wild-type | 0.001 |
| | | Dut | Empty vs. Wild-type | <0.001 |
| | | SFM | Empty vs. Y→F, K→R | 0.996 |
| | | Dut | Empty vs. Y→F, K→R | 0.993 |
| CWR-R1 (D) | RDH16 | SFM | Empty vs. Wild-type | 0.251 |
| | | Dut | Empty vs. Wild-type | 0.878 |
| | | SFM | Empty vs. Y→F, K→R | 0.206 |
| | | Dut | Emnty vs. Y→F, K→R | 0.212 |

Catalytic site deletion (Δcat), double mutation (Y→F, K→R)
Tukey-Kramer Adjusted P-values

Empty plasmid, or plasmids for over-expression of RDH16 wild-type or RDH16-Y176F,K180R, were expressed into VCaP, C4-2 or CWR-R1 cells and DHT levels were measured using LC-MS/MS. DHT levels were similar in VCaP cells in SFM treated with or without dutasteride, which suggested that dutasteride treatment did not lower VCaP DHT levels (FIG. 6B). Expression of wild-type RDH16 increased DHT levels (p=0.031) that were affected minimally by dutasteride treatment. RDH16-Y176F,K180R expression resulted in DHT levels similar to VCaP cells that contained the empty plasmid. C4-2 cells produced measurable levels of DHT that were reduced by dutasteride treatment (p=0.001; FIG. 6C; Table 10). Expression of wild-type RDH16 increased DHT levels (p=0.001) that appeared to be reduced by dutasteride treatment. C4-2 cells that expressed RDH16-Y176,K180R produced DHT levels similar to C4-2 cells with empty plasmid. RDH16 did not appear to increase DHT levels in CWR-R1 cells and dutasteride did not lower DHT levels of CWR-R1 cells that contained empty plasmid or expressed wild-type RDH16 (FIG. 6D). DHT levels appeared lower when CWR-R1 cells that expressed RDH16-Y176F, K180R were treated with dutasteride.

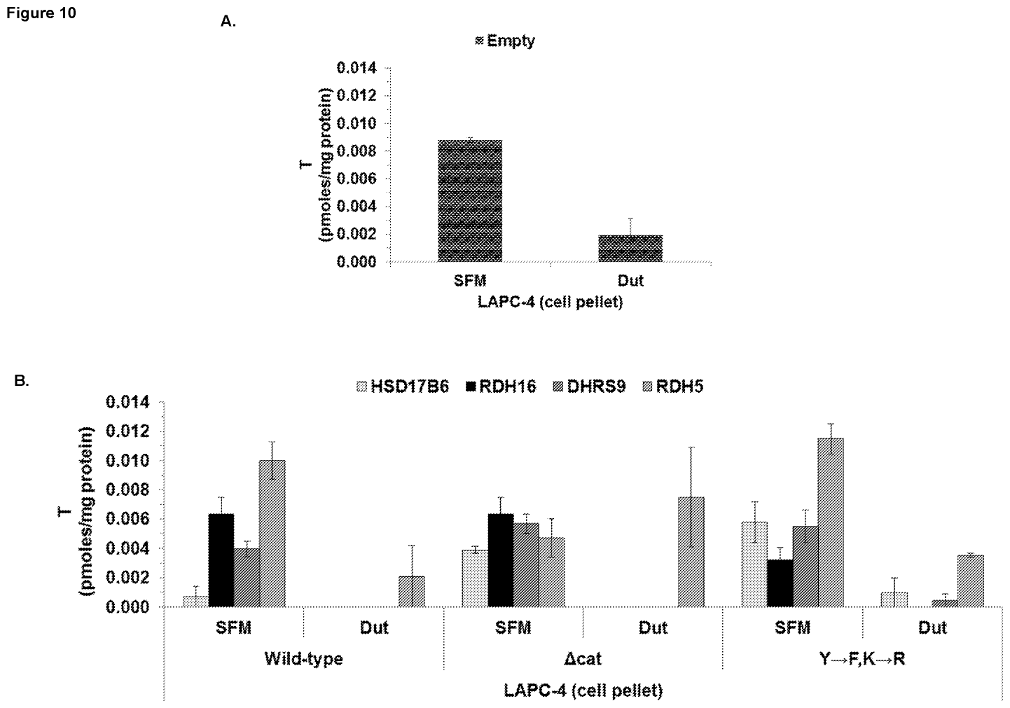

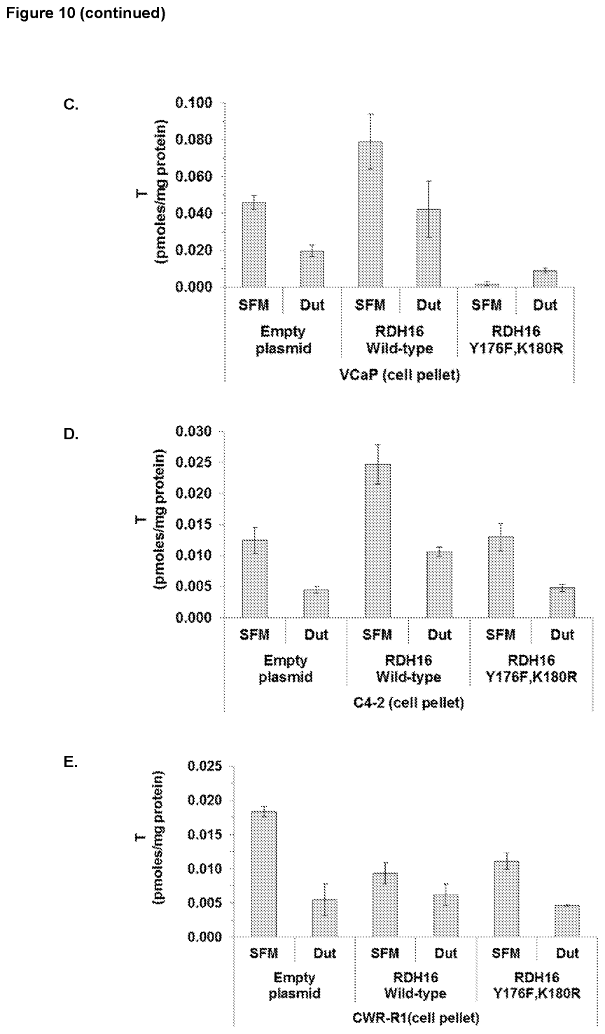

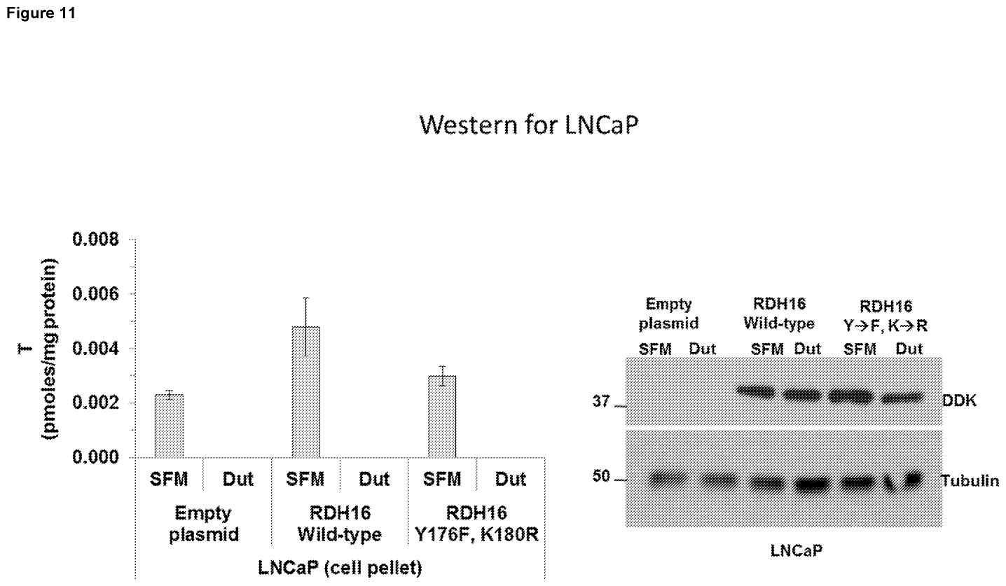

Western blot analysis demonstrated wild-type, Δcat or Y→F, K→R mutant 3α-oxidoreductase expression in the cell lines (FIG. 6E-H). LC-MS/MS revealed that dutasteride lowered T levels in all LAPC-4 cells, except in LAPC-4 cells that expressed RDH5-Δcat (FIG. 10). LNCaP cells did not have measurable DHT, and T was not detected after dutasteride treatment (FIG. 11).

### Discussion

The findings provide a proof of principal that 3α-oxidoreductases enhance DHT synthesis in CaP cells and inhibition of 3α-oxidoreductases impairs the ability of CaP cells to synthesize DHT using the primary backdoor pathway (FIG. 6I). The present data shows that 3α-oxidoreductases are expressed in specimens of AS-BP, AS-CaP and CRPC and AS-BP and AS-CaP after finasteride treatment. The primary backdoor pathway may facilitate DHT synthesis to overcome abiraterone treatment for CRPC and finasteride treatment for benign prostate enlargement or CaP chemoprevention. LC-MS/MS data provided evidence that all four 3α-oxidoreductases have similar enzymatic activity and that amino acid residues Y176/175 and K180/179 are essential for catalytic activity. Combination treatment with dutasteride and 3α-oxidoreductase mutation decreased DHT levels more effectively than dutasteride or 3α-oxidoreductase mutants alone. The studies demonstrate that 3α-oxidoreductase expression may provide AS-BP, AS-CaP and CRPC with a mechanism for resistance to SRD5A inhibitors that primarily block frontdoor DHT synthesis.

Experiments could not be performed using endogenous 3α-oxidoreductases because enzyme expression was so low in CaP cell lines. Therefore, 3α-oxidoreductases were expressed transiently in CaP and CV-1 cells. Androgen levels among replicates as determined using our LC-MS/MS analytical system were consistent across nine analytical runs conducted over a twenty month period. Western blot analysis showed expression levels were consistent among wild-type enzymes. Western blots also showed 3α-oxidoreductase mutant expression was lower than the expression of wild-type 3α-oxidoreductases that suggested amino acid substitutions may impair post-translational modification. However, sufficient enzyme was present based on the reproducibility of the replicates and differences In androgen metabolism observed among CaP cells transfected with control, wild-type or mutant 3α-oxidoreductases.

LC-MS/MS results were reliable over time and across replicates because the extracted calibration standards and plasma quality controls used to control and assess the quality of each LC-MS/MS analysis had an overall mean accuracy of 100% and 97.9%, respectively for the androgens analyzed in CaP cell pellets and media over nine independent sets of experiments. The LC-MS/MS method used for these studies was limited to six androgens. However, glucuronidated or sulfated androgens or androgen metabolites with activity in the glucocorticoid pathway were not measured. The LC-MS/MS method could not measure DIOL because DIOL did not display a mass spectrum sufficiently different from other androgens. Therefore, AND conversion to 5α-dione was used to measure individual enzyme activity for the expression studies in CV-1 cells. The studies suggested that the conversion of AND to 5α-dione is important clinically. The combination of dutasteride treatment and 3α-oxidoreductase mutation showed DHT levels were suppressed for 12 h. Clinical effectiveness will require longer studies of any potential inhibitor of the four 3α-oxidoreductases alone or in combination with dutasteride.

Taken together, the data [1] show that 3α-oxidoreductases are expressed in AS-BP, AS-CaP and CRPC; [2] demonstrate the catalytic residues necessary for the terminal step of the primary backdoor pathway, DIOL to DHT, are essential to all four 3α-oxidoreductases; and [3] provide evidence that combined SRD5A and 3α-oxidoreductase blockade lowered DHT levels more effectively than inhibition of either enzyme family alone. Inhibitors against the 3α-oxidoreductases are not yet available for clinical use and need to be identified. A new treatment strategy to block the key enzymatic steps of the frontdoor and primary and secondary backdoor pathways may decrease DHT levels more effectively than ADT alone. Further reduction of tissue DHT by inhibiting the last step of intracrine DHT synthesis should improve the clinical response to ADT or induce re-remission of CRPC and improve survival of men with advanced CaP.

While the present invention has been described through embodiments, these are intended to be illustrative and routine modifications are intended to be within the scope of the disclosure.

1. A method of inhibition of growth of prostate cancer cells comprising inhibiting the activity of more than one 3α-oxidoreductase enzymes that share a common catalytic site and that convert androstanediol to DHT.

2. The method of claim 1, wherein the more than one enzymes are selected from the group consisting of HSD17B6, RDH16, DHRS9 and RDH5.

3. The method of claim 1, wherein the activity of all of the enzymes in inhibited.

4. The method of claim 1, further comprising contacting the cells with a therapeutic effective against prostate cancer.

5. The method of claim 4, wherein the therapeutic comprises dutasteride and/or abiraterone.

6. The method of claim 1, wherein the activity of the more than one more than one 3α-oxidoreductase enzymes is inhibited by contact with a composition comprising a single inhibitor.

7. Prostate cancer cells in which the activity of two or more of the 3α-oxidoreductase HSD17B6, RDH16, DHRS9 and RDH5 has been inhibited.
